(19) United States
(12) Patent Application Publication
(10) Pub. No.: US 2025/0268522 A1
(43) Pub. Date: Aug. 28, 2025

(54) CLOSED LOOP CARE SYSTEM BASED ON COMPLIANCE WITH A PRESCRIBED MEDICATION PLAN

(71) Applicant: Medtronic, Inc., Minneapolis, MN (US)

(72) Inventors: Kate G. Arnebeck, Shoreview, MN (US); Ryan D. Wyszynski, Oak Grove, MN (US); Nirav A. Patel, Dublin, OH (US); Shantanu Sarkar, Roseville, MN (US); Hyun J. Yoon, Vadnais Heights, MN (US); John E. Burnes, Blaine, MN (US); Trent M. Fischer, St. Paul, MN (US)

(21) Appl. No.: 18/856,493

(22) PCT Filed: Apr. 3, 2023
(86) PCT No.: PCT/IB2023/053367

§ 371 Date: Oct. 11, 2024

Related U.S. Application Data
(60) Provisional application No. 63/363,461, filed on Apr. 22, 2022.

(51) Int. Cl. A61B 5/00; A61B 5/0538; G16H 10/60; G16H 20/10; G16H 50/30

(52) U.S. Cl. CPC A61B 5/4833; A61B 5/0538; A61B 5/686; A61B 5/7264; A61B 5/7275; A61B 5/746; G16H 10/60; G16H 20/10; G16H 50/30

(57) ABSTRACT

A medical system can be configured to receive medication data for a patient, the medication data being indicative of compliance of the patient with a prescribed medication plan; determine a compliance metric for the patient with the prescribed medication plan based on the medication data, wherein the compliance metric is indicative of a degree of compliance of the patient with the patient with the prescribed medication plan; obtain, via one or more medical devices, values for one or more physiological parameters for the patient; and based on the compliance metric and the values for the one or more physiological parameters for the patient, determine a heart failure risk score for the patient.

### FIELD

The disclosure relates to medical devices and, more particularly, medical devices for detecting or monitoring heart conditions.

### BACKGROUND

A variety of medical devices have been used or proposed for use to deliver a therapy to and/or monitor a physiological condition of patients. As examples, such medical devices may deliver therapy and/or monitor conditions associated with the heart, muscle, nerve, brain, stomach or other organs or tissue. Medical devices that deliver therapy include medical devices that deliver one or both of electrical stimulation or a therapeutic agent to the patient. Some medical devices have been used or proposed for use to monitor heart failure or to detect heart failure events.

Heart failure (HF) is the most common cardiovascular disease that causes significant economic burden, morbidity, and mortality. In the United States alone, roughly 5 million people have HF, accounting for a significant number of hospitalizations. HF may result in cardiac chamber dilation, increased pulmonary blood volume, and fluid retention in the lungs. Generally, the first indication that a physician has of HF in a patient is not until it becomes a physical manifestation with swelling or breathing difficulties so overwhelming as to be noticed by the patient who then proceeds to be examined by a physician. This is undesirable since hospitalization at such a time would likely be required for a cardiac heart failure patient to remove excess fluid and relieve symptoms.

### SUMMARY

The closed loop system of this disclosure may improve the quality of life for patients with real time tracking of medication usage, the individual's response to that medication trend over time, and improved medication compliance. For example, as will be discussed in more detail below, a system of this disclosure may enable a clinician, such as a physician, to match a patient's medication usage and compliance with a prescribed medication plan with temporally correlated HF risk scores (HFRSs) that can inform clinicians and patients as to whether or not medication compliance is improving a patient's health status or whether or not a lack of compliance is worsening the patient's health status.

In contrast to clinic visit/stay based monitoring of a patient's HF condition, the techniques of this disclosure may be implemented by systems including an implantable medical device (IMD) and computing devices that can autonomously and continuously (e.g., on a periodic or triggered basis without human intervention) collect physiological parameter data and medication compliance data while the implantable medical device is subcutaneously implanted in a patient over months or years and perform numerous operations per second on the data data to enable the systems herein to determine HFRSs and alert interested parties of the same. Using techniques of this disclosure with an IMD may be advantageous when a physician cannot be continuously present with the patient over weeks or months to evaluate the physiological parameters and medication compliance and/or where performing the operations on the data described herein (e.g., application of a probability model) could not practically be performed in the mind of a physician.

In some examples, the techniques and systems of this disclosure may use a probability model to more accurately determine HFRSs based on physiological data collected by an IMD and medication compliance data. In some examples, the probability model is trained with a set of training instances, where one or more of the training instances comprise data that indicate relationships between various input data and HFRS. Because the probability model is trained with potentially thousands or millions of training instances, the probability model may reduce the amount of error in determining HFRSs. Reducing HFRS errors using the techniques of this disclosure may provide one or more technical and clinical advantages, such as increasing reliability of another device, user, and/or clinician on the accuracy of determining a patient's condition and improve resulting treatment of the patient and patient outcomes.

According to one example of this disclosure, a device includes a memory and processing circuitry coupled to the memory and configured to receive medication data for a patient, the medication data being indicative of compliance of the patient with a prescribed medication plan; determine a compliance metric for the patient with the prescribed medication plan based on the medication data, wherein the compliance metric is indicative of a degree of compliance of the patient with the patient with the prescribed medication plan; obtain, via one or more medical devices, values for one or more physiological parameters for the patient; and based on the compliance metric and the values for the one or more physiological parameters for the patient, determine a heart failure risk score for the patient.

According to another example of this disclosure, a method includes receiving medication data for a patient, the medication data being indicative of compliance of the patient with a prescribed medication plan; determining a compliance metric for the patient with the prescribed medication plan based on the medication data, wherein the compliance metric is indicative of a degree of compliance of the patient with the patient with the prescribed medication plan; obtaining, via one or more medical devices, values for one or more physiological parameters for the patient; and based on the compliance metric and the values for the one or more physiological parameters for the patient, determining a heart failure risk score for the patient.

According to another example of this disclosure, a computer-readable storage medium stores instructions that when executed by one or more processors cause the one or more processors to receive medication data for a patient, the medication data being indicative of compliance of the patient with a prescribed medication plan; determine a compliance metric for the patient with the prescribed medication plan based on the medication data, wherein the compliance metric is indicative of a degree of compliance of the patient with the patient with the prescribed medication plan; obtain, via one or more medical devices, values for one or more physiological parameters for the patient; and based on the compliance metric and the values for the one or more physiological parameters for the patient, determine a heart failure risk score for the patient.

According to one example of this disclosure, a device includes means for receiving medication data for a patient, the medication data being indicative of compliance of the patient with a prescribed medication plan; means for determining a compliance metric for the patient with the prescribed medication plan based on the medication data, wherein the compliance metric is indicative of a degree of compliance of the patient with the patient with the prescribed medication plan; means for obtaining, via one or more medical devices, values for one or more physiological parameters for the patient; and means for determining a heart failure risk score for the patient based on the compliance metric and the values for the one or more physiological parameters for the patient.

The summary is intended to provide an overview of the subject matter described in this disclosure. It is not intended to provide an exclusive or exhaustive explanation of the systems, device, and methods described in detail within the accompanying drawings and description herein. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 is an example diagram of a probability framework including evidence nodes from diagnostic states of various parameters and one parent node.

FIG. 2 is a block diagram illustrating an example system that includes medical device(s) used to obtain diagnostic states from the various parameters for use as evidence nodes.

FIG. 3 is a functional block diagram illustrating an example configuration of the external device of FIG. 2.

FIG. 4 is a functional block diagram illustrating an example framework for a probability model to determine health risk probabilities for a patient using evidence obtained from the system of FIG. 2.

FIG. 5 is a flow diagram illustrating an example process that may be performed by one or more medical devices (e.g., IMDs) and/or a computing device, in conjunction with probability model 19 of FIGS. 2-4, to determine a probability score with respect to the patient, in accordance with one or more techniques disclosed herein.

FIG. 6A illustrates a chart of individual parameters that may serve as evidence nodes to the probability model of FIG. 4.

FIG. 6B illustrates a sum of individual probability scores of FIG. 6A on the left and a chart of combined probability scores using a probability model based on various evidence nodes of FIG. 4 on the right.

FIG. 7 illustrates the environment of an example medical system in conjunction with the patient, including an example implantable medical device (IMD) used to determine parameters of the patient.

FIG. 8 is a functional block diagram illustrating an example configuration of an IMD of FIG. 7.

FIG. 9 is a conceptual side-view diagram illustrating an example IMD of the medical system of FIGS. 7 and 8 in greater detail.

FIG. 10 is a flow diagram illustrating an example method that may be performed by one or more medical devices and/or processing devices, in accordance with one or more techniques disclosed herein.

FIG. 11A is a perspective drawing illustrating an example IMD.

FIG. 11B is a perspective drawing illustrating another example IMD.

Like reference characters denote like elements throughout the description and figures.

### DETAILED DESCRIPTION

Medication is often used to stabilize heart failure (HF) patients, but obtaining and tracking patient compliance data is often difficult. Health care providers may, for example, prescribe for a patient a medication plan that includes types of medications to take, dosages to take, schedules for taking the medication, maximum frequencies for taking the medication, and other such guidelines for taking the medication. While some patients may fully comply with all the guidelines of their medication plan, other patients may deviate from the prescribed doses and schedules. Some patients may even quit taking their medication altogether. In order to effectively manage a patient's condition, a clinician needs to know if the patient has taken their medication in the manner prescribed and then determine how the medication, or lack thereof, is impacting the patients' clinical condition. Timely visibility into the impact that medication plan compliance is having on the patient's condition is often a gap in the patient's care.

Various systems and techniques exist for determining Heart Failure Risk Score (HFRS) that can be used for both generating alerts and for driving better patient outcomes. These systems typically include one or more medical devices (either wearable or implantable), a patient device, such as a smartphone, and a cloud-based computing system. The HFRSs are typically determined by the patient device or the cloud-based computing system based at least in part on various physiological parameters of the patient that are monitored over a period of time by the one or more medical devices. Overlaying this medical device data with medication data (type, amount, frequency, time of day taken) could provide valuable information to the clinician allowing the clinician to individualize care for each patient and improving quality of life for patients. This closed loop system may also help drive patient compliance with their medications and understand the effectiveness of medications prescribed.

According to the techniques of this disclosure, physician medication orders can be entered into the cloud computing system. The cloud-based computing system can the pass this information along to the patient device to allow the patient to better manage the type of medication, the amount of medication, the frequency of the medication, and the time of day the medication should be taken. In one example, a small sensor in a pill (e.g., activated when an outer coating is dissolved) could allow a medical device, e.g., either an implantable or wearable device, to determine which medication was taken and the time of day the medication was taken. This medication data can then be fed from the medical device to the patient device and/or the cloud-based computing system, such that the medication data can be factored into the HFRS. If the HFRS exceeds a threshold set by the physician, then the cloud-based computing system or patient device may send a message to the patient via text, email, or in app messaging to let the patient know what medicine should be taken and the amount. In another example, a WiFi-enabled automatic pill dispenser may be an optional part of the system that can be used by the patient to dispense the proper amount of medication. The WiFi-enabled automatic pill dispensers may then transmit to the patient device medication data indicating whether or not the patient has taken their medication so that the medication data can be included in the calculating an HFRS for the patient.

In other examples, the medication data may be obtained based on user input. For example, the patient, a caretaker, or other user may enter into the patient device when the patient has taken their medication (type, amount, and time), so that this medication data can be used in determining the HRFS.

Once the patient takes the medication, the patient device can be notified of the amount of medication taken and the time the medication was taken either via a marker in the medication itself that is picked up by the device, automatic confirmation from the pill dispenser, or manual confirmation by the patient in the app. The medication data from knowing whether or not the patient took the medication may then be transmitted from the patient device to the cloud-based computing device and used as an input into the HFRS for the patient, thus leading to an improved HFRS score and generally improving the care of the patient.

In some examples, the clinicians or caregivers may also opt in for alerts for when medication is not taken (or taken) via text, email, or in app notifications. In some examples, an alert may be sent to the patient, clinician and/or caregiver only if the medication has not been taken and may include continuous reminders until the medication is taken to help with compliance. In some examples, the automatic pill dispenser can alert the pharmacy and patient and/or caregiver when a refill is approaching and/or another Rx is needed.

Using the techniques of this disclosure, clinicians can see trends, via the cloud-based computing device, related to the patients' response to the medications. This data can be viewed by an individual patient or group of similar patients in the cloud network. This may provide valuable information to the clinician allowing them to individualize care for each patient and improving their quality of life.

This closed loop system may improve the quality of life for patients with real time tracking of medication usage, the individual's response to that medication trend over time, and improved medication compliance. For example, as will be discussed in more detail below, a system of this disclosure may enable a clinician, such as a physician, to match a patient's medication usage and compliance with a prescribed medication plan with temporally correlated HFRSs that can inform clinicians and patients as to whether or not medication compliance is improving a patient's health status or whether or not a lack of compliance is worsening the patient's health status. Furthermore, a system of this disclosure may also send reminders to a patient to comply with their prescribed medication plan and, moreover, may even provide temporary revisions to the prescribed medication plan to compensate for past non-compliance. Such temporary revisions may, for example, include a recommendation to take a dose of medicine at an off-schedule time or to increase a dosage of a particular medication.

In some examples, the prediction and/or probability modeling described herein for determining an HFRS may include Bayesian Belief Networks (BBN) or Bayesian machine learning (ML) models (these sometimes referred to as Bayesian Networks or Bayesian frameworks herein), Markov random fields, graphical models, artificial intelligence (AI) models (e.g., Naive Bayes classifiers), and/or other belief networks, such as sigmoid belief networks, deep belief networks (DBNs), etc. In other examples, the disclosed technology may leverage non-Bayesian prediction or probability modeling, such as frequentist inference modeling or other statistical models. In addition, known model-selection techniques, such as Bayesian information criterion (BIC) or Akaike information criterion (AIC), may be used to evaluate probability models prior to use.

In some examples, an integrated diagnostics model may be used to determine a number of criteria that are met. The criteria may include a parameter corresponding to a level of compliance of the patient with a prescribed medication plan as well as other criteria based on sensed or measured physiological parameters. For example, the probability model may determine that X of Y criteria have been met with respect to the parameters. In such examples, Y may be the maximum number of criteria possible given the particular configuration of parameters the probability model is using, and X may be a variable less than or equal to Y that increments based on the parameters meeting certain criteria. In an illustrative example, the probability model may increment X in response to determining that the patient has an impedance score indicating a high diagnostic state and as such, may increment X.

Processing circuitry may determine, from the respective parameter values, diagnostic states for each parameter. A subcutaneous tissue impedance score, for example, may be used to determine a diagnostic state for the subcutaneous tissue impedance. Processing circuitry may compare the subcutaneous tissue impedance score to one or more risk thresholds to determine a diagnostic state of high (H) risk, medium (M) risk, or low (L) risk, in some examples. In some examples, processing circuitry may determine a joint diagnostic state based on multiple parameters that are independent of one another.

In some examples, diagnostic states may include a finite number of potential diagnostic states for each parameter (e.g., very high, high, medium, low, very low, etc.). For example, the diagnostic states may include states of high risk, medium risk, or low risk, for each parameter. In some instances, one or more of the parameters can have a different number of potential diagnostic states (e.g., one state, two states, three states, or more), whereas other parameters may have a greater or lesser number of potential diagnostic states. For example, night heart rate (NHR) may have three diagnostic states (H, M, and L), whereas atrial fibrillation (AF) burden may have less than three diagnostic states (H and L). In other examples, diagnostic states may include a continuum or sliding spectrum of diagnostic state values, rather than discrete states.

Diagnostic states of the parameters may be independent for each parameter. For example, a diagnostic state for a first set of one or more parameters may be independent of diagnostic states associated with one or more other parameters. In some examples, the probability mode framework, such as a BBN framework, may include additional parameters, where the respective values of the parameters are conditionally independent of one another. In an example, a high night heart rate may indicate an increase in sympathetic tone associated with a worsening condition. In addition, a decrease in impedance could reflect an increase in retained fluid. In such examples, each of these parameters for impedance and night heart rate may provide indications of a heart failure event. In accordance with techniques of this disclosure, however, each of these conditionally independent parameters may provide stronger evidence when used together to predict an adverse health event.

In an illustrative example, two example parameters may be conditionally independent of one another in the absence of an adverse health event. To illustrate, in the presence of the adverse health event, such as an HF event or fluid overload, the two example parameter variables may be correlated or dependent on one another, but in the absence of the adverse health event, the two variables may change independently from one another, indicating that the two variables are conditionally independent of one another. For example, in some instances, respiration rate (RR) will increase, whereas subcutaneous impedance will decrease during an adverse health event, such as HF, because HF may be the cause of these changes but not an effect of such changes. For example, an increase in RR does not cause HF, whereas RR may increase as a result of HF. In another example, RR may increase when a patient has anemia, whereas subcutaneous impedance may not change either way as a result of the anemia.

In some examples, processing circuitry may identify diagnostic states for parameters that the processing circuitry has deemed relevant to the goal of the probability model. In a non-limiting example, if the goal is to determine the likelihood of a clinically-significant HF event (e.g., meriting a remote-care phone call or clinic visit or hospital admission) in the next 30 days, certain parameters are more relevant to that probability determination than others, whereas a probability model for determining the likelihood of a preeclampsia-onset event in the next 60 days may require more or less parameter inputs and may require a modified version of the previous probability model or a different probability model altogether.

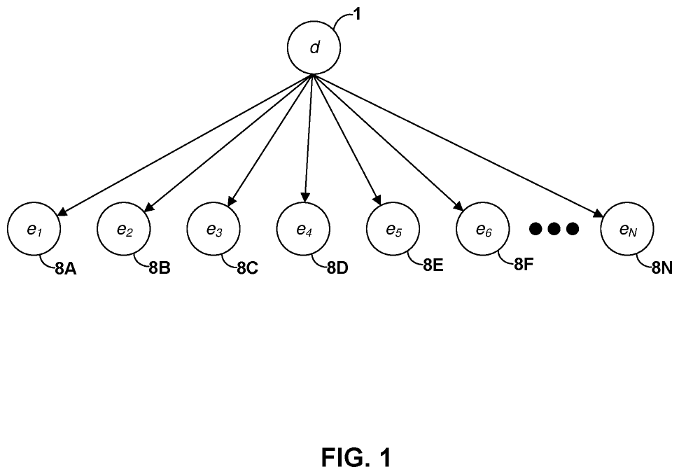

The diagnostic states may serve as evidence nodes for the probability model. FIG. 1 represents an example probability model framework that includes a parent node 1 and a plurality of evidence nodes 8A-8N (collectively, “evidence nodes 8”). Parent node 1 represents the posterior probability (e.g., the probability that an adverse health event is to occur based on diagnostic states of evidence nodes 8). In an example, the adverse health event may include a HF event, where d=H, for illustration purposes. The probability model may include any number of evidence nodes 8, as illustrated by evidence node 8N. Each of evidence nodes 8 may correspond to one or more parameters of a patient, including both physiological parameters and non-physiological parameters such as a level of compliance with a prescribed medication plan. As further discussed herein, each one of evidence nodes 8 may include a diagnostic state derived from one or more values that correspond to one or more parameters. In examples involving discrete states of d, the posterior probability for the occurrence of the HF event (d=H) may be expressed as:

$$P ( d = H \vert {e}_{1} , … , {e}_{N} ) = \frac{P ( d = H ) {\prod }_{i = 1}^{N} P ( {e}_{i} \vert d = H )}{{\sum }_{{d}_{1}} P ( d ) {\prod }_{i = 1}^{N} P ( {e}_{i} \vert d )}$$

In such examples, P(d) may represent a prior probability value, P(eᵢ|d) may represent a conditional likelihood parameter, d represents parent node 1, and e₁-e_{N} represent evidence nodes 8 in FIG. 1. Processing circuitry may determine the prior probability value and the conditional likelihood from existing parameter values prior to clinical event d in previous clinical study data. In some examples, the conditional likelihood parameter may assume, using previous probability data, what probability distribution is likely to exist, such that the processing circuitry can assume what probability scores are unlikely based on previous probability data. In some examples, the prior probability value may include a probability distribution absent any diagnostic states to use as evidence nodes. In other words, the prior probability value is what processing circuitry may believe at a particular point of time, whereas the posterior probability is what processing circuitry may believe in the presence of incoming diagnostic information.

In some examples, the probability score may include a joint probability distribution. In an example, for a n-node Bayesian network (where paᵢ is the parent node of node xi), the joint probability distribution may be expressed as:

$$P ( {x}_{i} , … , {x}_{n} ) = {\prod }_{i}^{N} P ( {x}_{i} \vert p {a}_{i} ) ,$$

For example, a posterior probability may involve determining joint probability distributions and defining multiple combinations of conditional probabilities. A probability model may provide a framework for assumptions regarding the explicit relationship between parameter values to make these determinations more feasible. For example, Bayesian theory may assign explicit relationships between parameter values in order to determine posterior probability scores from the various evidence nodes 8 in FIG. 1.

For example, a posterior probability may include a posterior distribution. In some cases, the posterior distribution may include a Gaussian distribution. In other cases, the posterior distribution may include a non-Gaussian distribution.

In some examples, processing circuitry may determine and/or utilize conditional likelihood tables, BBN tables, prior probability values, etc. For example, the conditional likelihood parameters may take the form of conditional likelihood tables defined for each diagnostic state for each parameter. The posterior probability may then be tabulated for all possible combinations of diagnostic states to determine a posterior probability, or in some instances, a probability table.

In some instances, a single evidence node may be derived from multiple parameters, such as with a Multi-Variable Node (MVN). In an example, MVNs may be based on multiple parameters, such as AF burden as a first parameter and ventricular rate values as a second parameter, where the parameters factor into a single evidence node.

Processing circuitry may use the evidence nodes as input to a probability model to determine a posterior probability score. In such instances, the posterior probability score indicates a likelihood that a patient will experience an adverse health event within a predetermined period of time (e.g., within 30 days of determining the probability score). As discussed herein, the probability model may use as additional inputs the prior probability value and a conditional likelihood parameter to determine the posterior probability score. In some examples, processing circuitry may then update the probability model using the determined posterior probability score.

In some examples, the probability score is compared to one threshold for each of one or more risk levels (e.g., high risk threshold, medium risk threshold, low risk threshold). In an example, the probability score may be compared to two thresholds to provide hysteresis in the alert decision. In such examples, an alert may be generated when the probability score crosses a first, higher threshold. The alert is ended when the probability score subsequently crosses a second, lower threshold. By generating alerts in this manner, a device may generate fewer “sporadic” alerts that may be misinterpreted by the patient or a clinician when the probability score fluctuates near the higher, alert threshold value. In addition, the device may provide hysteresis alerts for a plurality of risk levels. For example, medium and/or low risk alerts may also have hysteresis thresholds.

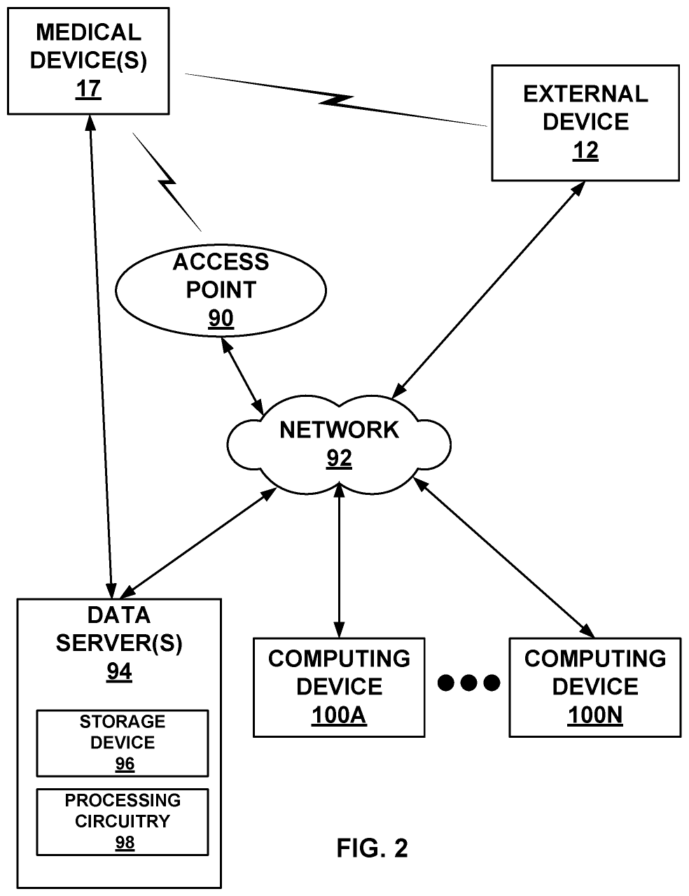

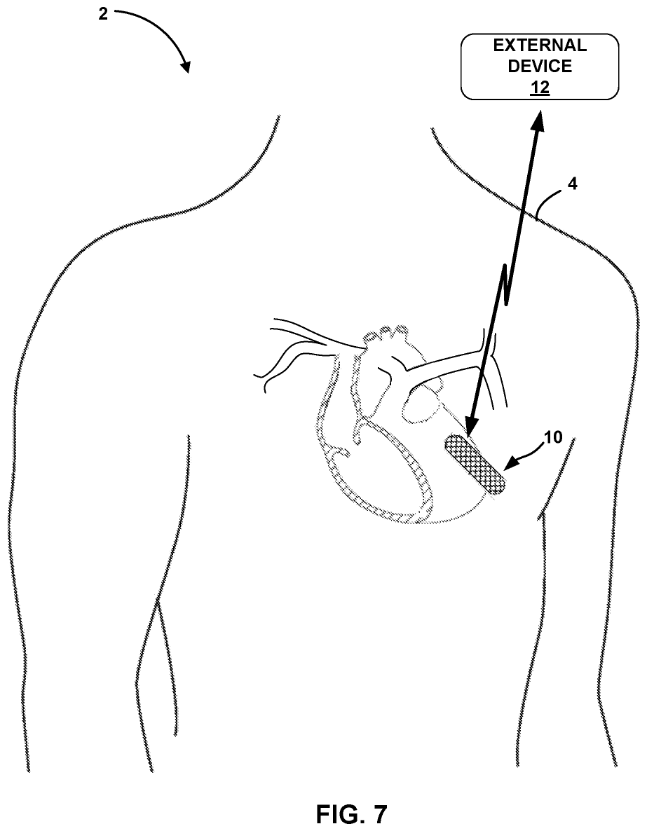

FIG. 2 is a block diagram illustrating an example system that includes one or more medical device(s) 17, an access point 90, a network 92, external computing devices, such as data servers 94, and one or more other computing devices 100A-100N (collectively, “computing devices 100”). In some examples, medical device(s) 17 may include an IMD, such as IMD 10 described with reference to FIGS. 7-9. In this example, medical device(s) 17 may use communication circuitry 54 to communicate with external device 12 via a first wireless connection, and to communicate with an access point 90 via a second wireless connection.

In one or more of the various example techniques described with reference to FIG. 2, access point 90, external device 12, data server(s) 94, and computing devices 100 may be interconnected and may communicate with each other through network 92. Network 92 may include a local area network, wide area network, or global network, such as the Internet. The example system described with reference to FIG. 2 may be implemented, in some aspects, with general network technology and functionality similar to that provided by the Medtronic CareLink® Network, developed by Medtronic, Inc., of Minneapolis, MN.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient.

Medical device(s) 17 may be configured to transmit data, such as sensed, measured, and/or determined values of parameters (e.g., heart rates, impedance measurements, impedance scores, fluid indices, respiratory rate, activity data, cardiac electrograms (EGMs), historical physiological data, blood pressure values, etc.), to access point 90 and/or external device 12. In some examples, medical device(s) 17 may be configured to determine multiple parameters. For example, medical device(s) 17 may include an IMD 10 configured to determine respiration rate values, subcutaneous tissue impedance values, EGM values. In such examples, IMD 10 may provide multiple parameters to serve as evidence nodes to the probability model 19. Access point 90 and/or external device 12 may then communicate the retrieved data to data server(s) 94 via network 92.

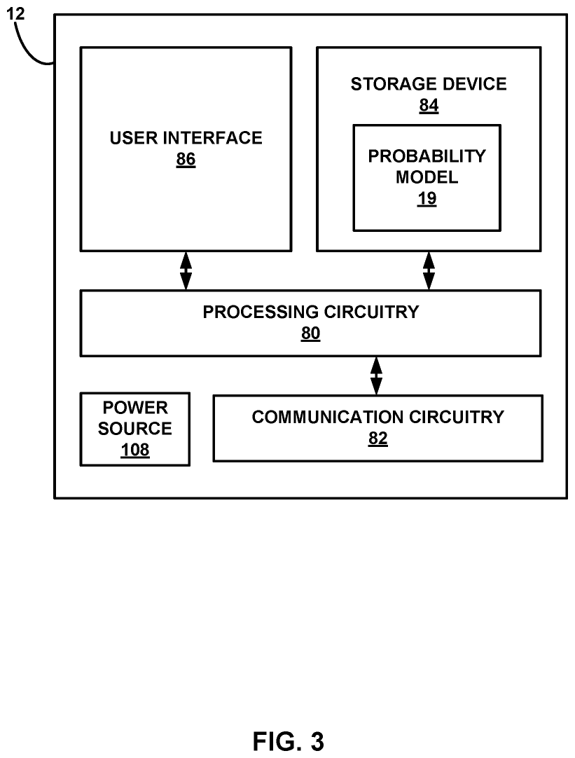

In some instances, one or more of medical device(s) 17 may transmit data over a wired or wireless connection to data server(s) 94 or to external device 12. For example, data server(s) 94 may receive data from medical device(s) 17 or from external device 12. In another example, external device 12 may receive data from data server(s) 94 or from medical device(s) 17, such as parameter values, diagnostic states, or probability scores, via network 92. In such examples, external device 12 may determine the data received from data server(s) 94 or from medical device(s) 17 and may store the data to storage device 84 (FIG. 3) accordingly.

In addition, one or more of medical device(s) 17 may serve as or include data server(s) 94. For example, medical device(s) 17 may include enough storage capacity or processing power to perform the techniques disclosed herein on a single one of medical device(s) 17 or on a network of medical device(s) 17 coordinating tasks via network 92 (e.g., over a private or closed network). In some examples, one of medical device(s) 17 may include at least one of the data server(s) 94. For example, a portable/bedside patient monitor may be able to serve as a data server, as well as serving as one of medical device(s) 17 configured to obtain physiological parameter values from patient 4. In other examples, data server(s) 94 may communicate with each of medical device(s) 17, via a wired or wireless connection, to receive physiological parameter values or diagnostic states from medical device(s) 17. In a non-limiting example, physiological parameter values may be transferred from medical device(s) 17 to data server(s) 94 and/or to external device 12.

In some cases, data server(s) 94 may be configured to provide a secure storage site for data that has been collected from medical device(s) 17 and/or external device 12. In some instances, data server(s) 94 may include a database that stores medical- and health-related data. For example, data server(s) 94 may include a cloud server or other remote server that stores data collected from medical device(s) 17 and/or external device 12. In some cases, data server(s) 94 may assemble data in web pages or other documents for viewing by trained professionals, such as clinicians, via computing devices 100. One or more aspects of the example system described with reference to FIG. 2 may be implemented with general network technology and functionality, which may be similar to that provided by the Medtronic CareLink® Network.

In some examples, one or more of computing devices 100 may be a tablet or other smart device located with a clinician, by which the clinician may program, receive alerts from, and/or interrogate medical device(s) 17. For example, the clinician may access data collected by medical device(s) 17 through a computing device 100, such as when patient 4 is in between clinician visits, to check on a status of a medical condition. In some examples, the clinician may enter instructions for a medical intervention for patient 4 into an application executed by computing device 100, such as based on a status of a patient condition determined by medical device(s) 17, external device 12, data server(s) 94, or any combination thereof, or based on other patient data known to the clinician.

One computing device 100 may transmit instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 (or relay an alert determined by a medical device 17, external device 12, or data sever 94) based on a probability score (e.g., posterior probability) determined from parameter values of patient 4, which may enable patient 4 proactively to seek medical attention prior to receiving instructions for a medical intervention. The parameter values of patient 4 may include one or more physiological parameters of patient 4 as well as a level of compliance of patient 4 with a prescribed medication plan. In this manner, patient 4 may be empowered to take action, as needed, to address his or her medical status, which may help improve clinical outcomes for patient 4.

In the example illustrated by FIG. 2, data server(s) 94 includes a storage device 96 (e.g., to store data retrieved from medical device(s) 17) and processing circuitry 98. Although not illustrated in FIG. 2 computing devices 100 may similarly include a storage device and processing circuitry. Processing circuitry 98 may include one or more processors that are configured to implement functionality and/or process instructions for execution within data server(s) 94. For example, processing circuitry 98 may be capable of processing instructions stored in memory 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of data server(s) 94 and/or the processing circuitry of computing devices 100 may implement any of the techniques described herein to analyze physiological parameters received from medical device(s) 17 and other parameters such as a level of compliance of the patient with the prescribed medication plan, e.g., to determine a probability score of patient 4.

In some examples, storage device 96 of data server(s) 94 may store a probability model 19. In some examples, external device 12 may store probability model 19. For example, data server(s) 94 may transmit probability model 19 to external device 12, where external device 12 may store the probability model 19 in a memory device of external device 12 (not shown in FIG. 2). External device 12 and/or data server(s) 94 may use the probability model 19 to determine a probability score with respect to a health risk for patient 4. External device 12 and/or data server(s) 94 may also store, or otherwise obtain, a prescribed medication plan for the patient. The prescribed medication plan may, for example, be based on a physician's medication orders, which can be uploaded from a computing device of the physician to data server(s) 94 of external device 12 via network 92.

Processing circuitry 98 of data server(s) 94 and/or the processing circuitry of computing devices 100 may also retrieve instructions for utilizing a selected probability model (e.g., one selected using a known selection technique) and execute the probability model to determine the probability score. Processing circuitry 98 of data server(s) 94 and/or the processing circuitry of computing devices 100 may retrieve such data and instructions from storage device 96 or in some instances, from another storage device, such as from one of medical devices 17.

Storage device 96 may include a computer-readable storage medium or computer-readable storage device. In some examples, memory 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

According to one example, processing circuitry may obtain medication data indicative of compliance of a patient with a prescribed medication plan. The processing circuitry may, for example, be processing circuitry from one or more of external device 12, data servers 94, or computing devices 100. The processing circuitry may obtain the medication data from external device 12 or data servers 94. To obtain the medication data for the patient, the processing circuitry (e.g., external device 12) may solicit user input indicating the compliance with the prescribed medication plan. In other examples, the processing circuitry may use one or more automated techniques for determining if the patient is complying with the prescribed medication plan. The prescribed medication plan may, for example, indicate medication types, dosages, and a schedule prescribed, or otherwise recommended, by a doctor.

The processing circuitry may determine a compliance metric for the patient with the prescribed medication plan based on the medication data. The processing circuitry may determine the compliance metric based on a frequency with which the patient takes the medication relative to an expected frequency established by the prescribed medication plan, based on a measurement of a deviation from times in which the patient takes the medication relative to expected times established by the prescribed medication plan, based on an amount of medication taken during a time period relative to an expected amount of medication taken during the time period as established by the prescribed medication plan, based on a frequency which medication is taken during a time period relative to an expected frequency of medication taken during the time period as established by the prescribed medication plan, or some other such criteria.

The processing circuitry may also, obtain, via medical device(s) 17, values for one or more physiological parameters for the patient. The one or more physiological parameters for the patient may include posture, respiratory effort, respiratory rate, temperature, short term HRV, R-wave amplitude, an interval or morphological metric for one or more heart sounds, nighttime rest versus daytime active body angle, chronotropic incompetence, B-type natriuretic peptide (BNP), renal dysfunction, blood pressure, or any other such physiological parameter. The processing circuitry determines a heart failure risk score for the patient, in accordance with techniques described herein, based on the compliance metric and the values for the one or more physiological parameters for the patient.

The processing circuitry may store, in data server(s) 94, for example, a plurality of heart failure risk scores and corresponding timestamps for the patient. The processing circuitry may also store, in data server(s) 94, a plurality of amounts of compliance with corresponding timestamps for the patient. The processing circuitry may match, using the first corresponding timestamps and the second corresponding timestamps, the heart failure risk scores to temporally correlated amounts of compliance. As the effect of complying or not complying with a medication plan may take time to manifest in the patient, the heart failure risk scores and temporally correlated amounts of compliance may not correspond to the same moments in time. A heart failure risk score may, for example, lag its temporally correlated compliance metric by some period of time, such as 1-7 days.

Using data stored in data server(s) 94, the processing circuitry may compare a trend of the plurality of heart failure risk scores for the patient to a trend of the temporally correlated plurality of amounts of compliance for the patient to determine an effect that a level of compliance of the patient with the prescribed medication plan is having on a health status of the patient. The processing circuitry may, for example, determine based on the heart failure risk scores and the temporally correlated amounts of compliance, whether a level of compliance of the patient with the prescribed medication plan is affecting a health status of the patient. In this context, the health status of the patient may be the same as the heart failure risk score or may be some other metric determined at least in part based on the heart failure risk score.

FIG. 3 is a block diagram illustrating an example configuration of components of external device 12. In some examples, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as one of medical device(s) 17 (e.g., IMD 10). Under the control of processing circuitry 80, communication circuitry 82 may receive downlink telemetry from, as well as send uplink telemetry to, one of medical device(s) 17 (e.g., IMD 10), or another device (e.g., data server(s) 94). Communication circuitry 82 may be configured to transmit or receive signals via inductive coupling, electromagnetic coupling, near-field communication (NFC) technologies (e.g., inductive coupling, NFC or other communication technologies operable at ranges less than 10-20 cm), RF communication, Bluetooth®, Wi-Fi™, or other proprietary or non-proprietary wireless communication schemes. Communication circuitry 82 may also be configured to communicate with devices other than medical device(s) 17 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Storage device 84 may store one or more probability models 19. Storage device 84 may also store historical data, diagnostic state data, parameter values, probability scores, etc.

Data exchanged between external device 12 and medical device(s) 17 may include operational parameters (e.g., physiological parameter values, diagnostic states, etc.). External device 12 may transmit data including computer readable instructions which, when implemented by medical device(s) 17, may control medical device(s) 17 to change one or more operational parameters and/or export collected data (e.g., physiological parameter values). For example, processing circuitry 80 may transmit an instruction to medical device(s) 17 which requests medical device(s) 17 to export collected data (e.g., impedance data, fluid index values, and/or impedance scores, blood pressure, ECG records, etc.) to external device 12.

In turn, external device 12 may receive the collected data from medical device(s) 17 and store the collected data in storage device 84. Processing circuitry 80 may implement any of the techniques described herein to model parameter values to determine diagnostic states, probability scores, etc. Using the modeling techniques disclosed herein, processing circuitry 80 may determine a likelihood that the patient is experiencing an adverse health event (e.g., heart failure decompensation) or is likely to experience an adverse health event within a predetermined amount of time (e.g., within the next 3 days, 7 days, 10 days, 30 days, 40 days, etc.). In an illustrative example, the predetermined amount of time may be at least approximately 7 days from when the probability score is determined, such that the probability score indicates the likelihood that an adverse health event will occur in the next 7 days or indicates that the patient is likely already experiencing an adverse health event, such as heart failure decompensation.

External device 12 may be a computing device with a display viewable by a user and an interface for providing input to external device 12 (i.e., a user input mechanism). The user may be a physician technician, surgeon, electrophysiologist, clinician, or patient 4. In some examples, external device 12 may be a notebook computer, tablet computer, computer workstation, one or more servers, cellular phone, personal digital assistant, handheld computing device, networked computing device, or another computing device that may run an application that enables the computing device to interact with IMD 10. External device 12 is configured to communicate with IMD 10 and, optionally, another computing device, via wired or wireless communication. External device 12, for example, may communicate via NFC technologies (e.g., inductive coupling, NFC or other communication technologies operable at ranges less than 10-20 cm) and far-field communication technologies (e.g., Radio Frequency (RF) telemetry according to the 802.11 or Bluetooth® specification sets, or other communication technologies operable at ranges greater than NFC technologies). In some examples, external device 12 may include a programming head that may be placed proximate to the body of patient 4 near the IMD 10 implant site in order to improve the quality or security of communication between IMD 10 and external device 12.

In one example, a user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to medical device(s) 17 (e.g., cardiac EGMs, blood pressure, subcutaneous impedance values, RR, etc.). In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both. Using any of the various mechanisms described herein, a user may use user interface 86 to input data indicative of a compliance of patient 4 with a prescribed medication plan.

In some examples, user interface 86 of external device 12 may receive input from the user. The user interface may include, for example, a keypad and a display, which may for example, be a cathode ray tube (CRT) display, an LCD, or an LED display. The keypad may take the form of an alphanumeric keypad or a reduced set of keys associated with particular functions. External device 12 can additionally or alternatively include a peripheral pointing device, such as a mouse, via which the user may interact with the user interface. In some examples, a display of external device 12 may include a touch screen display, and a user may interact with external device 12 via the display. It should be noted that the user may also interact with external device 12 remotely via a networked computing device.

Power source 108 delivers operating power to the components of external device 12. Power source 108 may include a battery and a power generation circuit to produce the operating power. In some embodiments, the battery may be rechargeable to allow extended operation. Recharging may be accomplished by electrically coupling power source 108 to a cradle or plug that is connected to an alternating current (AC) outlet. In addition or alternatively, recharging may be accomplished through proximal inductive interaction between an external charger and an inductive charging coil within external device 12. In other embodiments, traditional batteries (e.g., nickel cadmium or lithium ion batteries) may be used. In addition, external device 12 may be directly coupled to an alternating current outlet to power external device 12. Power source 108 may include circuitry to monitor power remaining within a battery. In this manner, user interface 86 may provide a current battery level indicator or low battery level indicator when the battery needs to be replaced or recharged. In some cases, power source 108 may be capable of estimating the remaining time of operation using the current battery.

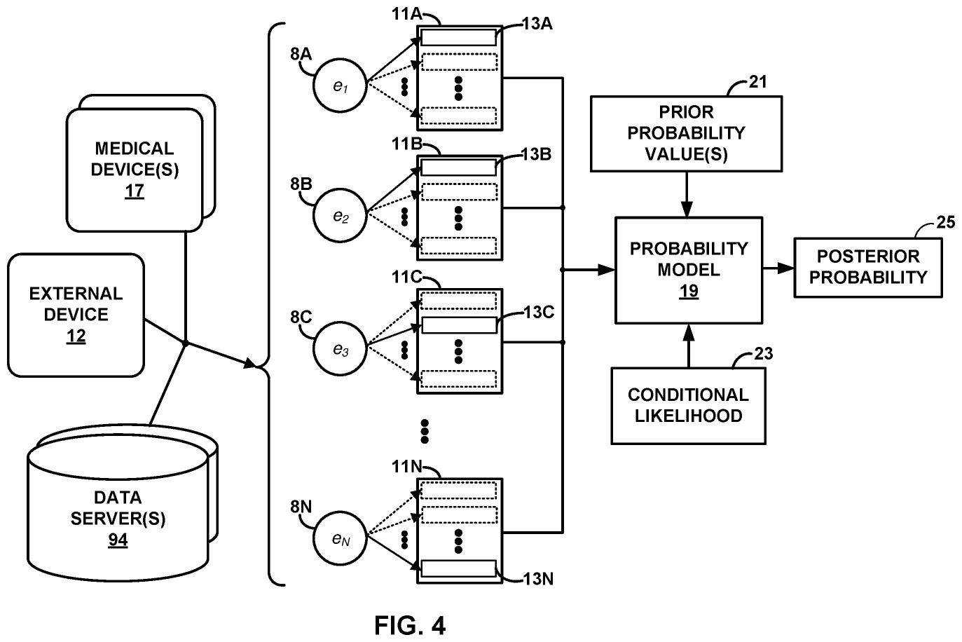

FIG. 4 illustrates a framework that uses a medical system, such as system 2, to monitor health events and the likelihood of health events of patient 4. The medical system may include external device 12 or one or more of data server(s) 94. Although primarily described in terms of one or more data server(s) 94 determining the probability score, it will be understood that any one or more devices (e.g., processing circuitry of such devices), such as external device 12, one or more medical devices 17, or computing devices 100, may perform the probability determination using probability model 19 as described herein. In any event, FIG. 4 illustrates external device 12, medical device(s) 17, and/or data server(s) 94 as being configured to supply input to probability model 19.

In some examples, storage device 96 of data server(s) 94 may store the parameter values that relate to one or more parameters, which may have been received from one or more other devices of the system via network 92. Data server(s) 94 may store the parameter values as raw data or as conditioned data via signal processing techniques. For example, data server(s) 94 may store, within storage device 96, medication data indicative of a compliance of patient 4 with a prescribed medication plan. In some examples, processing circuitry 98 of data server(s) 94 determines a compliance metric for patient 4 with the prescribed medication plan based on the medication data. In some examples, processing circuitry of the medical device(s) or processing circuitry 80 of external device 12 determines the compliance metric.

Processing circuitry, e.g., processing circuitry 98 of data server(s) 94, may store data received from medical device(s) 17 (e.g., from IMD 10) to a storage device, e.g., storage device 96 of data server(s) 94. In an example, storage device 96 may be configured to store indications of whether patient 4 took or did not take an expected dose of medication, a time at which patient 4 took a dose of medication, an amount of medication patient 4 took, or other such medication data. In such examples, processing circuitry 98 of data server(s) 94 may determine the compliance metric, which may be an input to probability 19 or used to determine inputs to probability model 19. In some examples, medical device(s) 17, e.g., IMD 10, or external device 12 or another device may determine the compliance metric.

Processing circuitry 98 of data server(s) 94 may determine one or more compliance metrics based on medication data. In some examples, data server(s) 94 may also receive one or more physiological parameters from IMD 10 or medical devices 17. In any case, data server(s) 94 may receive data from medical device(s) 17 and determine, via probability model 19, a probability score based on the data.

With reference still to FIG. 4, processing circuitry 98 of data server(s) (or processing circuitry of any other device of the system) may, in some examples, perform the probability score determination using probability model 19 in accordance with the following. As described herein, processing circuitry 98 may be coupled to one or more storage devices such that processing circuitry 98 may leverage the various data repositories in order to determine the probability score.

In some examples, processing circuitry 98 may be configured to determine a respective one or more values for each of a plurality of parameters. For instance, processing circuitry may determine one or more values for a first parameter and one or more values for a second parameter. In one example, the values may correspond to measurement readings determined via medical device(s) 17, measurement readings determined by IMD 10, or a compliance metric determined by any of medical devices 17, external device 12, or data server(s) 94. For example, in addition to a compliance metric, the values for physiological parameters may include respiration rate values, ECG values, activity level values, etc. For example, the values may indicate at when patient 4 was active or when patient 4 was inactive. The values may include accelerometer values that indicate a posture of patient 4 or a change in the posture of patient 4 over time (e.g., a posture-change count). Posture change count may be based on z-axis accelerometer values. Other values may include periodic x, y, and z-axis accelerometer measurements.

In some examples, processing circuitry 98 may adjust the criteria for determining each diagnostic state as more information becomes available to processing circuitry 98 over time. For example, processing circuitry 98 may determine that one or more diagnostic states for one or more parameters may be optimized. In some examples, processing circuitry 98 may determine such optimization potential based on the output of a ML model trained on posterior probability data, diagnostic states, and criteria performance data. In any event, processing circuitry 98 may adjust the criteria used (right-hand column below in Table 1) for determining the one or more diagnostic states for one or more parameters.

In an illustrative and non-limiting example of how diagnostic states may be determined, Table 1 below shows diagnostic states in the left-hand column and criteria values (e.g., parameter values) in the right-hand column. The criteria values provided below are merely to illustrate an example of how diagnostic states may correlate to criteria for determining a particular diagnostic state. Likewise, the parameters below are provided as example parameters, and in some examples, other parameters may additionally or alternatively be used, such as heart sounds, stepping out of bed count, or other parameters disclosed herein.

TABLE 1

| Diagnostic | Parameter Values |
|---|---|
| state | and Criteria |
| Medication | |
| Compliance | |
| H | Doses missed in time period ≥ 2 |
| M | Doses missed in time period = 1 |
| L | Doses missed in time period = 0 |
| Impedance | |
| score | |
| H | ImpScore ≥ 7 |
| M | ImpScore ≥ 1 and ImpScore ≤ 6 |
| L | ImpScore = 0 |
| RR | |
| H | maxRR₇ ≥ 22 brpm OR cvarRR₃₀ ≥ 23 OR ND (RR₃₀ > 20 brpm) ≥ 16 |
| M | minRR₃₀ > 11.5 brpm and {Not ‘H’} |
| L | {Not ‘H’ OR ‘M’} |
| AF | |
| H | [{avgAFB₇ > 1 hr OR ND(AFB₃₀ > 6 hrs) ≥ 2} AND ND(AFB₃₀ > 23 hrs) < 30] |
| | OR max2minAFB₇ > 3.5 hr OR |
| | max2minAFB₃₀ > 4.5 hr OR ND(AFB₃₀ > 6 hrs AND VRAF₃₀ ≥ 90 bpm) ≥ 1 |
| | OR ND(AFB₃₀ > 23 hrs AND VRAF₃₀ ≥ 90 bpm) ≥ 1 |
| L | Not ‘H’ |
| NHR | |
| H | ND(NHR₇ ≥ 85 bpm) ≥ 3 OR maxNHR₇ > 100 bpm OR avgNHR₃₀ > 90 bpm |
| | OR avgNHR₇ > 90 bpm OR avgDHR-NHR₇ <− 1 bpm |
| M | {ND(NHR₃₀ ≥ 85 bpm) ≥ 3 OR maxNHR₃₀ > 85 bpm OR maxNHR₇ > 85 bpm |
| | OR minNHR₃₀ > 65 bpm OR avgDHR- |
| | NHR₇ < 3 bpm} AND Not ‘H’ |
| L | {Not ‘H’ OR ‘M’} |
| HRV | |
| H | ND(HRV₃₀ ≤ 60 ms) ≥ 25 OR max2avgHRV₃₀ ≥ 85% OR minHRV₇ < 35 ms |
| | OR CSFRHRV₃₀ <− 12 |
| M | {ND(HRV₇ ≤ 60 ms) ≥ 2 OR ND(HRV₃₀ ≤ 60 ms) ≥ 6 OR max2avgHRV₃₀ |
| | ≥65% OR minHRV₃₀ < 55 ms OR avgHRV₃₀ < 65 ms |
| | OR avgHRV₇ < 75 ms OR CSFRHRV₇ <− 2} AND Not ‘H’ |
| L | {Not ‘H’ OR ‘M’} |
| ACT | |
| H | ND(ACT₇ ≤ 30 min) ≥ 7 OR ND(ACT₃₀ ≤ 30 min) ≥ 27 OR CSFRACT₇ < −43 |
| | OR avgACT₇ < 10 min |
| M | {ND(ACT₃₀ ≤ 30 min) ≥ 11 OR max2avgACT₃₀ ≥ 150% OR CSFRACT₃₀ |
| | <− 3 OR avgACT₃₀ < 30 min} AND Not ‘H’ |
| L | Not ‘H’ OR ‘M’ |

where ND=number of days; bpm=beats per minute; CSAR=Cumulative Sum Adaptive Reference, IMP=impedance; CSFR=Cumulative Sum Fixed Reference, Thr=Threshold; AdapThr=Adaptive Threshold; Subscripts indicate preceding timeframe (e.g., look-back window) size.

In some examples, the impedance score (ImpScore) may be determined as follows:

The following elements add a score of 1:
CSAR-IMP₃₀≥0.6*AdapThr for ≥1 day
CSAR-IMP₇≥0.6*AdapThr for ≥1 day
CSAR-IMP₃₀≥1.7*AdapThr for ≥1 day
CSAR-IMP₇≥1.7*AdapThr for ≥1 day
CSAR-IMP₃₀≥3.2*AdapThr for ≥1 day
CSAR-IMP₇≥1.5*AdapThr for ≥7 days
   And the following elements add a score of 2:
CSARIMP₇≥3.2*AdapThr for ≥1 day
CSARIMP₃₀≥1.5*AdapThr for ≥24 days
AvgIMP₇≤600Ω
AvgIMP₃₀≤600Ω

Referring to Table 1 above, patient 4 may have a diagnostic state for a single medication, a diagnostic state for multiple medications, or multiple diagnostic states for multiple medications. Examples of such medications include hypertension medications, diuretic medications, ACE-I, beta blockers, or other such medications. The diagnostic state for medication compliance may be based on a number of doses that patient 4 misses in a time period. In the example above, a diagnostic state of H may correspond to 2 or more doses missed in a time period, and a diagnostic state of M may correspond to 1 dose missed in the time period. A diagnostic state of L may correspond to no doses missed in the time period. For certain medications, the missed doses may be weighted. As one example, for certain medications, a missed morning dose may be more detrimental to patient 4 than a missed evening dose.

The time period may, for example, be a day, two days, a week or any other such time period. It is contemplated that diagnostic states for different medications may be based on different numbers of doses missed for different time periods. Some medications may have more or fewer corresponding diagnostic states. The diagnostic state may also be adjusted upwards or downwards depending on if patient 4 misses multiple doses in a row or otherwise close in time or if patient 4 misses intermittent doses. The diagnostic states for different medications may adjust differently. For example, missing a few doses of a diuretic medication may more significantly increase a risk to patient 4 than missing a few doses of a lipid lowering medication.

Throughout this disclosure a day may generally refer to a 24-hour period. For example, a day may refer to midnight to midnight or some other 24-hour period. The same may generally be said about a week, month, or other time period.

As described herein, the plurality of parameters may include one or more subcutaneous tissue impedance or electrical parameters identified from the one or more subcutaneous tissue impedance measurements. The one or more subcutaneous tissue impedance parameters may include a subcutaneous tissue impedance score, as well as fluid index values. In some examples, the subcutaneous tissue impedance parameters may include subcutaneous tissue impedance measurements, fluid index values, statistical representations of subcutaneous tissue impedance measurements, respiration rate, etc. In some examples, fluid index values may be derived from other sensors, such as intra-cardiac pressure sensors. For example, an intra-cardiac pressure sensor may detect higher pressures, which may be indicative of a higher amount of fluid. The cardiac pressure data may be used to compute one or more fluid index values and/or scores based on fluid index values.

In some examples, the parameters include at least one value corresponding to a RR of patient 4. The RR of patient 4 may be determined in a number of different ways (e.g., chest wall movement, acute changes in subcutaneous fluid with respiratory cycle induced venous return changes, etc.). In some examples, processing circuitry 80 is configured to identify, based on the one or more subcutaneous tissue impedance measurements, a periodic variation (e.g., increase and decrease) in subcutaneous tissue impedance. In such examples, processing circuitry 98 is configured to determine, based on the periodic variation (e.g., increase and decrease) in subcutaneous tissue impedance, the RR of patient 4. In some examples, one of medical device(s) 17 may determine RR in accordance with other techniques, such as by using R-wave amplitude changes or changes in R-R intervals. For example, one of medical device(s) 17 may use R-R interval parameters (e.g., R-wave to R-wave interval) to determine the RR of patient 4. R-R intervals may be derived from ECG measurements. In another example, one of medical device(s) 17 may determine RR from accelerometer or optical sensors, e.g., photoplethysmography (PPG) sensors.

In such examples, IMD 10 or processing circuitry 98 may utilize the impedance measurements to determine a RR of patient 4. The subcutaneous tissue impedance values may include low-frequency fluctuations that correspond to the RR. Subcutaneous impedance is sensitive to conductivity of fluid around IMD 10. With each inhalation, the intrathoracic pressure reduces increasing pulmonary blood volume. An increase in pulmonary blood volume tends to lead to a reduction of pulmonary artery pressure. A reduction of pulmonary artery pressure tends to cause a reduction of right arterial pressure and an increase of venous return. This may cause a reduction of extracellular/extravascular volume and thus, an increase in impedance. The opposite sequence of events leads to reduction of impedance with exhalation. In another example, a movement of the chest wall of patient 4 may lead to changes in measured impedance, as well.

In some examples, processing circuitry 98 may be configured to identify diagnostic states 11A-11N (collectively, “diagnostic states 11”) for each of the parameters based on the respective values. For example, various thresholds may be used to determine a diagnostic state of a parameter. In some examples, processing circuitry 80 is configured to select, from at least three potential diagnostic states, a single diagnostic state for each of evidence nodes 8. For example, the diagnostic state may be high, medium, low, where some parameters may include more or less diagnostic states (H and L). In the example described with reference to FIG. 4, the diagnostic states are selected from N number of diagnostic states. In a few examples, diagnostic state 13A has a first diagnostic state (e.g., high or very high), where diagnostic state 13C has a second diagnostic state, where the first diagnostic state may include a high risk categorization and the second diagnostic state may include a medium risk categorization depending on the respective one or more parameter values. Further, diagnostic state 13B corresponding to evidence node 8B may be high or very high and diagnostic state 13N corresponding to evidence node 8N may be low or very low.

The diagnostic states may be determined independently for each parameter. For example, processing circuitry 98 may compare the values obtained for a first parameter to one or more thresholds to determine a diagnostic state for the first parameter independently of processing circuitry comparing values obtained for a second parameter to one or more thresholds to determine a diagnostic state for the second parameter. In some examples, data server(s) 94 may receive the diagnostic state for one or more of the diagnostic states 11. In other examples, data server(s) 94 may determine the diagnostic state for one or more of the diagnostic states 11 based on the respective values of the parameters. In any event, diagnostic states 11 define evidence nodes 8 for probability model 19. In other words, diagnostic states 11 serve as evidence nodes 8 for probability model 19.

In some examples, in addition to a medication compliance metric, the parameters may include physiological parameters such as long-term HRV, NHR, ACT, AF, or ventricular rate. Moreover, the parameters may include posture, respiratory effort, temperature, short term HRV, R-wave amplitude, heart sound, nighttime rest versus daytime active body angle, chronotropic incompetence, B-type natriuretic peptide (BNP), renal dysfunction, or blood pressure. In addition, parameters may further include RR interval, posture-change count and accelerometer data values. In any event, the parameters may also include subcutaneous tissue impedance parameters from IMD 10 or another medical device 17.

In some examples, probability model 19 may include a Bayesian framework or BBN. Other suitable probability models may be used to determine probability scores given diagnostic states of parameters. For example, a Bayesian ML model may be used to determine probability scores based on diagnostic states of parameters. Processing circuitry may train probability model 19 on values associated with the parameters. In other examples, processing circuitry may include as input to probability model 19 prior probability value(s) 21 or conditional likelihood 23. For example, processing circuitry 98 may determine, from the plurality of parameters, prior probability value 21. The prior probability value 21 may be determined from existing data. Processing circuitry 98 may also determine, from the plurality of parameters, conditional likelihood parameter 23.

In some examples, processing circuitry 98 may determine the conditional likelihood, or P(eᵢ|d), from existing data. Processing circuitry 98 may utilize existing data from one or more patients or subjects, where the existing data is then used to determine conditional likelihood parameters of a model utilizing a probability theorem, such as Bayes rule.

In one example, the value of ‘d’ may represent the presence or absence of an HF event or other adverse health event. As such, processing circuitry 98 may use earlier data to determine whether a particular diagnostic criterion was satisfied before an HF event (e.g., P(eᵢ|d=1)) or whether the particular diagnostic criterion was satisfied when there was no HF event (e.g., P(eᵢ|d=0)). In an example, using a first evidence node e₁ as corresponding to ‘impedance score’, processing circuitry 98 may determine from a plurality of existing data points the conditional likelihood for: P(e₁=H|d=1), P(e₁=H|d=0), P(e₁=M|d=1), P(e₁=M|d=0), P(e₁=L|d=1), and P(e₁=L|d=0). That is, processing circuitry 98 may determine the conditional likelihood from data derived from True Positives, False Positives, False Negatives, and False Positives. In some examples, processing circuitry 98 use the same data to provide a desired sensitivity and specificity of HF detection. That is, posterior probability 25 may represent an estimate of positive predictive value (PPV) based on sensitivity, specificity and event rate (e.g., prior probability 21).

Processing circuitry 98 may determine the conditional likelihoods for each parameter used as an input evidence node to probability model 19 (e.g., each of e;). In some examples, processing circuitry 98 may then utilize the conditional likelihood probabilities to determine the probability model 19. In such examples, the determined probability model 19 may include a computable joint distribution.

As such, processing circuitry 98 may identify prior probability value 21 and/or the conditional likelihood parameter 23 as inputs to probability model 19 when determining the probability score. In such examples, the probability model may be expressed as:

$$P ( d , {e}_{1} , … , {e}_{N} ) = P ( d ) {\prod }_{i = 1}^{N} P ( {e}_{i} \vert d ) ,$$

wherein P(d) represents the prior probability value, P(eᵢ|d) represents the conditional likelihood parameter, d represents a parent node, and e₁-e_{N} represent the evidence nodes.

In some examples, processing circuitry 98 may be configured to determine a probability score from probability model 19 based on evidence nodes 8. In an example, the BBN may have one or more child nodes (e.g., n-nodes as shown in FIG. 4) and a parent node, represented by posterior probability 25.

The probability score may include a likelihood that the patient is experiencing an adverse health event or is likely to experience the adverse health event within a predetermined amount of time. In an example, the adverse health event could be a worsening HF event (e.g., HF decompensation). The probability score may be expressed in terms of a percentage, a decimal number, or a threshold categorization, such as 50%, 0.5, or medium likelihood, where in this example, 50% corresponds to a threshold categorization of medium likelihood. In some examples, the probability score may be expressed in terms of a range such as >50% or between 50-60%.

In some examples, processing circuitry 98 may determine the probability score for predetermined amount of time in the future. This may be known as a look-forward period. In some examples, the predetermined amount of time is approximately 30 days relative to when the probability score is determined. For example, the probability score may indicate that patient 4 has a 50% chance of experiencing an adverse health event in the next 30 days. In some instances, the predetermined amount of time may be more or less than 30 days depending on the particular configuration of probability model 19. As such, probability model may determine a probability score that indicates the likelihood of an adverse health event, such as a heart failure worsening event, occurring within the predetermined timeframe (e.g., next 30 days).

Processing circuitry 98 may, in some instances, determine the predetermined amount of time, such that the predetermined amount of time serves as a buffer period. In other words, at the end of the predetermined amount of time (e.g., 30 days), processing circuitry 98 may determine another probability score using data received during a preceding timeframe (e.g., the last 30 or 60 days). Processing circuitry 98 may perform automatic probability determinations using probability model 19 after the predetermined amount of time and after each buffer period thereafter. In other examples, processing circuitry 98 may determine a probability score in response to receiving a command signal (e.g., from a user via a user interface). Processing circuitry 98 may alter the predetermined timeframe slightly to account for the different days in a month. For example, approximately 30 days may include 31 days, 29 days, or 28 days, for convenience of patient 4. That is, patient 4 may have an easier time tracking buffer periods based on months or weeks rather than based on strict 30-day periods.

In some examples, processing circuitry, e.g., processing circuitry 50 of IMD 10, processing circuitry 80 of external device 12, or processing circuitry 98 of data server(s) 94, may determine the probability score on a daily basis. For example, processing circuitry 98 may determine the probability score every day based on data corresponding to a previous X number of days. In some examples, processing circuitry 98 may store in storage device 96 diagnostic states for various parameters each day for a finite number of days, such as in a first in, first out (FIFO) buffer or sliding window. In some examples, processing circuitry 98 may store the last 30 diagnostic states for each parameter determined on a daily basis for the past 30 days. For example, processing circuitry 98 may store the last 30 diagnostic states for impedance scores determined on a daily basis for the past 30 days, store the last 30 diagnostic states for RR determined on a daily basis for the past 30 days, etc. Processing circuitry 98 may determine the probability score at a predefined time interval each day using the previous 30 diagnostic states of each parameter determined over the past 30 days as input to the probability model 19. In another example, processing circuitry 98 may determine the probability score at a predefined time interval each day using the previous 15 diagnostic states of each parameter determined over the past 15 days as input to the probability model 19. In any event, processing circuitry 98 may receive data from medical device(s) 17 on a periodic basis, such as on a daily, weekly, or biweekly basis, etc. In such examples, processing circuitry 98 may determine the probability score upon receiving the data from medical device(s) 17 according to the periodic transmission rate of medical device(s) 17 (e.g., daily, weekly, biweekly, etc.). That is, in one example, processing circuitry 98 may determine diagnostic states (e.g., risk states) for each parameter. In such examples, processing circuitry 98 may combine the last X number of days of diagnostic states together to determine a probability score using probability model 19.

In another example, processing circuitry 98 may determine the probability score (e.g., risk score) and diagnostic states on a periodic basis. In addition, processing circuitry 98 may determine the status of the health condition of patient 4 using the probability score and a threshold on a periodic basis. In a non-limiting example, processing circuitry 98 may compute the probability score, diagnostic states, and status on a daily basis. In such examples, processing circuitry 98 may store the probability score and/or diagnostic state for the last X number of days, such as for the last 30 days. In some examples, processing circuitry 98 may determine the probability score on a day basis using diagnostic data from the past X number of days, such as the last 30 days. In such examples, processing circuitry 98 may determine, on any given day, that the probability score satisfies a threshold. For example, processing circuitry 98 may determine that the probability score exceeds a threshold. In such examples where processing circuitry 98 determines that probability score satisfies a threshold, processing circuitry 98 may transmit an alert externally, such as to a physician device or patient device.

Although described with reference to processing circuitry 98, the techniques of this disclosure are not so limited. In some examples, other processing circuitry (e.g., processing circuitry 80, processing circuitry 50, or processing circuitry of another one of medical device(s) 17, such as a CPU of one of medical device(s) 17) may perform one or more of the techniques of this disclosure and may coordinate with other devices accordingly. For example, processing circuitry of one of medical device(s) 17 may determine the probability score on a daily basis, compare the probability score to a threshold, and cause the transmission of an alert where the probability score satisfies the threshold. In such examples, a particular medical device from one of medical device(s) 17 may receive data (e.g., diagnostic data) from network 92, such as from other medical device(s) 17, external device 12, or data server(s) 94, and may determine the probability score using processing circuitry included with the particular medical device.

In examples where storage device 96 does not have data corresponding to a particular timeframe, processing circuitry 98 may extrapolate data or interpolate data or in some examples, processing circuitry 98 may determine an extent to which the data for a parameter is missing and determine whether to use the parameter when determining the probability score, as discussed herein.

In another example, processing circuitry 98 may determine the probability score based on determined values of the parameters that correspond to a preceding timeframe relative to when the probability score is determined. For example, processing circuitry 98 may identify diagnostic states based on parameter values determined during a preceding timeframe prior to determining the probability score. In one example, the preceding timeframe may be approximately 30 days relative to when the probability score is determined. In some examples, the preceding timeframe and the predetermined amount of time may include the same amount of time relative to when the probability score is determined. For example, where the predetermined amount of time is 30 days in the future, the preceding timeframe may be 30 days of past data, and where the predetermined amount of time is 29 days in the future, the preceding timeframe may be 29 days in the past.

In some examples, the amount of time for each of the predetermined amount of time and the preceding timeframe may be different based on the number of days in each month. For example, processing circuitry 98 may determine a probability score on the last day of each month and for convenience, may forecast for the next month based on the parameters determined for the preceding month, in which case the predetermined amount of time and the preceding timeframe may include a different amount of time. In other examples, the amount of time for each may remain constant regardless of convenience factors (e.g., 60 days on either end, or 30 days for the predetermined amount of time in the future and 45 days for the preceding timeframe).

In some examples, the amount of time for the preceding timeframe may be dependent on values associated with the parameters. For example, processing circuitry 98 may determine diagnostic states for subcutaneous tissue impedance scores on a 30-day preceding timeframe basis, whereas processing circuitry 98 may determine diagnostic states for respiration rate on a shorter or longer preceding timeframe basis. In such examples, processing circuitry 98 may use a plurality of preceding timeframes for various parameters. In other examples, processing circuitry 98 may use a common preceding timeframe regardless of any resolution parameters used to determine the parameter values (e.g., medication compliance, impedance scores, NHR, etc.), where resolution parameters may include filters, time constraints, or activity determinations.

With reference still to FIG. 4, processing circuitry 98 is configured to identify, from the respective one or more values for each parameter, a plurality of parameter features that encode amplitude, out-of-normal range values, and temporal changes. In the example of amplitude, a physiological parameter feature may encode R-wave amplitudes, accelerometer signal amplitudes, etc. For example, processing circuitry 98 may determine whether a particular parameter satisfies an absolute threshold. In an illustrative example, processing circuitry 98 may determine whether an average NHR of patient 4 is greater than a predefined threshold of 90 bpm. In the example of out-of-range values, a parameter feature may encode use range values to determine whether a parameter includes out-of-range values to encode. For example, processing circuitry 98 may determine a high heart rate based on expected heart rate values.

In an illustrative example, processing circuitry 98 may determine NHR out-of-range values by comparing the average NHR to determine how many data NHR has been greater than 90 bpm or less than 55 bpm. In the example of temporal changes, a parameter feature may encode changes in a parameter over time. In one example, processing circuitry 98 may encode a feature of subcutaneous impedance measurements with changes in impedance over a period of days or weeks. Similar to calculating the fluid index using impedance values, processing circuitry 98 may determine relative changes in a parameter value to determine temporal changes, rather than absolute changes. In an illustrative example, processing circuitry 98 may determine whether an average or current-day NHR value has increased in a sustained manner over the last 7 days or 30 days relative to NHR values in the last 7 days or 30 days.

In such examples, processing circuitry 98 is configured to identify the evidence nodes based at least in part on the plurality of parameter features. For example, processing circuitry 98 may extract features that encode information regarding out-of-normal range values, as well as temporal changes at weekly and monthly time scale for the parameters. In a non-limiting example, processing circuitry 98 may determine diagnostic categories based on a combination of features, such that the HF hospitalization (HFH) rates increase from one category to the next. For example, the category with the largest number of HFH rates may be designed to have the lowest occurrence rate through the feature extraction process.

In some examples, processing circuitry 98 is configured to identify a plurality of parameter features based on the respective one or more values for each parameter. The parameter features are configured to, upon analysis, yield a same number of potential diagnostic states for each parameter. In some examples, the same number of potential diagnostic states may be three potential diagnostic states (e.g., H, M, and L). In other examples, one or more parameters may have a different number of potential diagnostic states, such as one or two potential diagnostic states. For example, AF may have two diagnostic states of high and low. In such examples, processing circuitry 98 is configured to identify, from the potential diagnostic states, the diagnostic state for each of the parameters.

Processing circuitry 98 may extract features from the parameters and/or from the parameter values. For example, processing circuitry 98 may analyze a large set of time series data for each parameter for time windows including the number of days the values are outside a normal amplitude range, cumulative sum of difference between the raw measurement and an adaptive reference (CSAR), cumulative sum of difference between the raw measurement in a fixed reference (CSFR), number of days CSAR or CSFR were above a threshold, slope or rate of change of raw measurement values, or mean, median, minimum, and maximum measurement values. Processing circuitry 80 may extract such features for each parameter to encode amplitude and temporal characteristics with respect to particular temporal scales.

In some examples, processing circuitry 98 may determine a MVN as one of the evidence nodes. For instance, multiple parameters may factor into determining a single child node of evidence nodes 8. In a non-limiting example, processing circuitry 98 is configured to determine an input to a first child node of evidence nodes 8 based on a combination of one or more values. For example, one evidence node may be based on a combination of an indication of AF extent in patient 4 during a time period and one or more values indicating a ventricular rate during the time period (e.g., during AF). In addition, processing circuitry 98 may be configured to determine an input to a second child node of the plurality of evidence nodes based on the respective one or more values of the one or more subcutaneous tissue impedance parameters. In such instances, evidence node 8A may include a combination of an AF extent indication value(s) and ventricular rate value(s), whereas evidence node 8A may indicate one or more subcutaneous tissue impedance parameter values (e.g., subcutaneous tissue impedance score, fluid indices, etc.). Evidence node 8B may, for example, include medication data indicative of compliance of the patient with a prescribed medication plan. In this context, compliance may include both affirmative compliance and a lack of compliance.

With reference still to FIG. 4, processing circuitry 98 may determine, for each of the plurality of parameters or evidence nodes 8, the respective one or more parameter values at various frequencies. For example, processing circuitry 98 may determine the values for evidence node 8A at a different frequency than for evidence node 8B. Thus, diagnostic states 11 may update at different frequencies. In such examples, processing circuitry 98 may delay execution of probability model 19 until an appropriate number of diagnostic states are deemed current or updated. In any event, processing circuitry 80 may determine the diagnostic states using the respective one or more values. Processing circuitry 98 may use the diagnostic states to determine posterior probability 25. Processing circuitry 98 may then store, the respective one or more values and/or the probability score to, as examples, storage device 96, storage device 84, and/or storage device 56 of medical device(s) 17 (e.g., IMD 10).

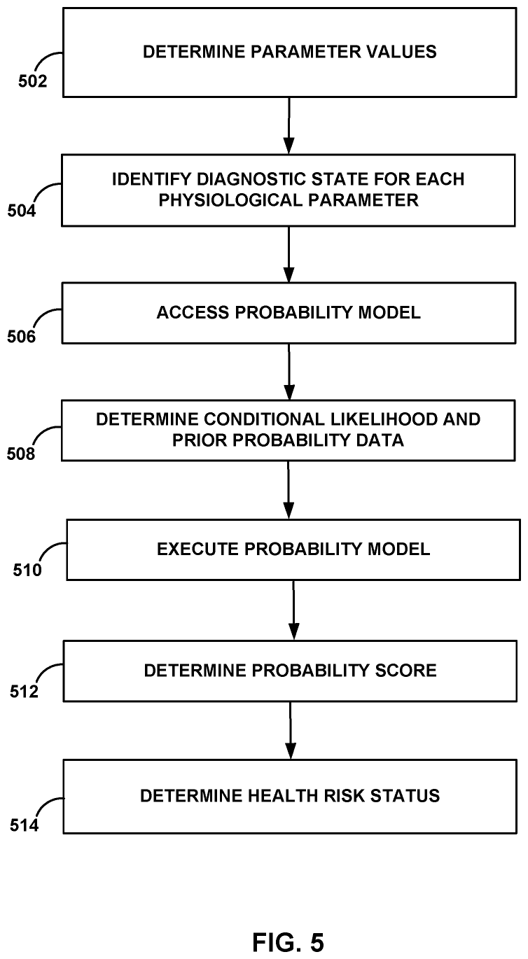

FIG. 5 illustrates an example method that may be performed by one or more of medical devices 17, external device 12, and/or data server(s) 94 in conjunction with probability model 19 described with reference to FIGS. 2-4, to determine a probability score with respect to the patient, in accordance with one or more techniques disclosed herein. Although described as being performed by data server(s) 94, one or more of the various example techniques described with reference to FIG. 5 may be performed by any one or more of IMD 10, external device 12, or data server(s) 94, e.g., by the processing circuitry of any one or more of these devices.

In some examples, processing circuitry, e.g., processing circuitry 50 of IMD 10, processing circuitry 80 of external device 12, or processing circuitry 98 of data server(s) 94, may determine values of parameters, such as those parameters described herein (502). For each of the parameters, processing circuitry 98 may identify diagnostic states 11 (504). In some instances, processing circuitry 98 may filter irrelevant parameters or parameter values prior to identifying diagnostic states or after identifying diagnostic states. For example, certain parameters or values thereof may not be relevant to the purpose of deploying probability model 19. In such instances, those parameters or values may still be available to processing circuitry 80, but processing circuitry 98 may determine those parameters should not be used as evidence nodes 8. At any point in time, processing circuitry 98 may access probability model 19 (506). For example, processing circuitry 98 may access probability model 19 stored in storage device 96.

In some examples, processing circuitry 98 may determine conditional likelihood and prior probability data (508). For example, the conditional likelihood parameters may take the form of conditional likelihood tables defined for each diagnostic state for each parameter. The posterior probability may then be tabulated for all possible combinations of diagnostic states to determine a posterior probability.

In some examples, processing circuitry 98, or other processing circuitry, may have previously used prior probability value(s) 21 and conditional likelihood 23, e.g., based on prior data of one or more subjects, to create probability model 19. For example, probability model 19 may include a look-up table (LUT) that the processing circuitry generated using prior probability value(s) 21 and conditional likelihood 23. That is, probability value(s) 21 and conditional likelihood 23 may be used to create probability model 19 but not to determine posterior probability 25 for a given patient.

Processing circuitry 98 may then execute probability model 19 (510). Processing circuitry 98 may execute probability model 19 in response to a user command or may do so automatically in response to a triggering event. For example, processing circuitry 98 may determine that all necessary diagnostic states have been determined and that the probability model 19 is ready for execution. In some examples, processing circuitry 98 may receive data for various parameters or processing circuitry 98 may access data from storage device 96. Processing circuitry 98 may determine the diagnostic state (L/M/H) for each parameter. For the particular set of parameters used (e.g., parameters having sufficient data), processing circuitry 98 may use the diagnostic states to map to a particular row of the LUT. Processing circuitry 98 may identify the posterior probability 25 that is mapped to the particular row. Processing circuitry 98 may subsequently utilize the data that was used to determine the posterior probability 25 to train the probability model 19 for future rounds of determining probability scores. For example, processing circuitry 98 may use the current or incoming data to determine the prior probability value(s) 21.

In some examples, processing circuitry 98 may execute probability model a number of times in a finite period of time in order to determine an average probability score for the finite period of time. In some instances, processing circuitry 98 may execute probability model in accordance with a Monte Carlo simulation (e.g., using repeated random sampling to obtain numerical results).

As such, processing circuitry 98 may determine one or more probability scores from probability model 19 (512). Processing circuitry 98 may use evidence nodes 8 as input to probability model 19 to determine a posterior probability score that indicates a likelihood that a patient will experience an adverse health event within a predetermine period of time (e.g., within 30 days of determining the probability score). As discussed herein, the probability model may use the prior probability value and a conditional likelihood parameter as additional inputs to determine the posterior probability score. The probability score (p-value) may be represented as a decimal value. In some examples, processing circuitry may then update the probability model using the determined posterior probability score. In some examples, a Bayesian ML model may determine the probability score, where the ML model may be trained on prior probability values and feedback received regarding the accuracy of the probability score in predicting adverse health events.

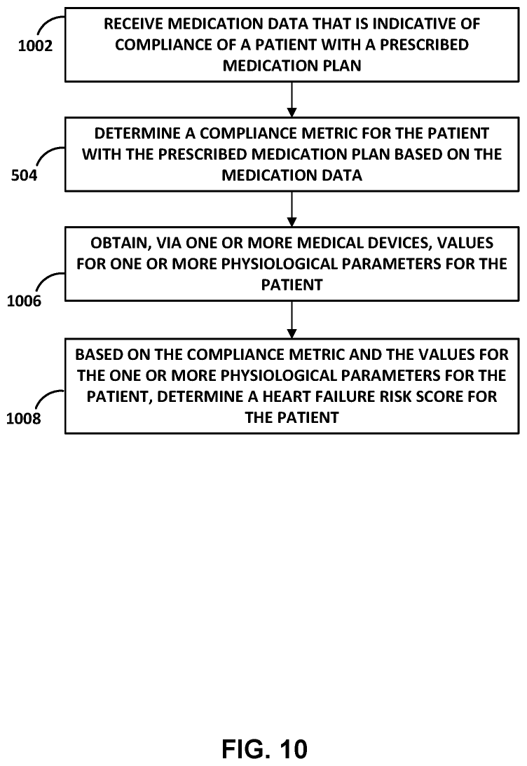

As discussed in more detail with reference to FIG. 10, processing circuitry 98 may compare the probability score to at least one risk threshold. Based on the comparison, processing circuitry 98 may determine one of plurality of discrete risk categorizations. For example, discrete risk categorizations may be high risk, low risk, or medium risk. Although described as being performed by server 94, one or more of the various example techniques described with reference to FIG. 5 may be performed by any one or more of IMD 10, external device 12, or server 94, e.g., by the processing circuitry of any one or more of these devices. In this way, processing circuitry 98 may determine a health risk status for a patient based at least in part on the probability score (514). In an example, processing circuitry 98 may be configured to monitor a patient, for example, with heart failure for a developing heart failure decompensation in accordance with one or more of the various techniques disclosed herein to then allow for early intervention potentially before clinical symptoms are experienced or hospitalization is needed.

It should be noted that one or more of the various example techniques described with reference to FIG. 5 may be performed on a periodic basis. For example, the probability score may be determined according to a resolution parameter setting of medical device(s) 17 and/or for patient 4. In other examples, the probability score may be calculated irrespective of the resolution parameter. Data server(s) 94 may calculate the probability score once a day, each week, every two weeks, each month, etc. In some examples, data server(s) 94 may also calculate the probability score in response to a user command (e.g., from a physician, from a user interface) or in response to a satisfaction of another condition, such as upon receiving or determining a particular number of diagnostic states, or an indication of a change in the condition of patient from a source other than the application of the probability model.

Data server(s) 94 may also trigger a probability score determination when an activity level or other parameter of patient 4 satisfies a threshold (e.g., low activity when patient 4 is resting or sleeping). In one other example, data server(s) 94 or medical device 17, e.g., IMD 10, may determine the probability score of patient 4 on a per measurement basis, such as on a per impedance score determination basis. A person of skill in the art would appreciate that various periods may exist for when IMD 10 or external device 12 may determine a probability score in accordance with FIG. 5, transmit a probability score, etc. In some examples, data server(s) 94 may determine the probability score of patient 4 in response to receipt of parameter data for patient via network 92, e.g., from medical device(s) 17.

In addition, although described in terms of data server(s) 94 performing one or more of the various example techniques of this disclosure, it is to be understood that any number of different components described with reference to, for example, FIGS. 4, 5, 7, and 8, and combinations thereof, may perform one or more of the various example techniques of this disclosure. For example, IMD 10 or external device 12 may determine diagnostic states 11 or parameter values. In addition, data server(s) 94 may include multiple computing devices (e.g., a remote cloud server) that collectively determines probability scores of patient 4 experiencing an adverse health event within a predetermined period of time. In addition, it is to be understood that the example components described with reference to FIGS. 4, 5, 7, and 8 may perform some or all of the example techniques described with reference to FIGS. 4, 5, and 10 in parallel or in conjunction with one another.

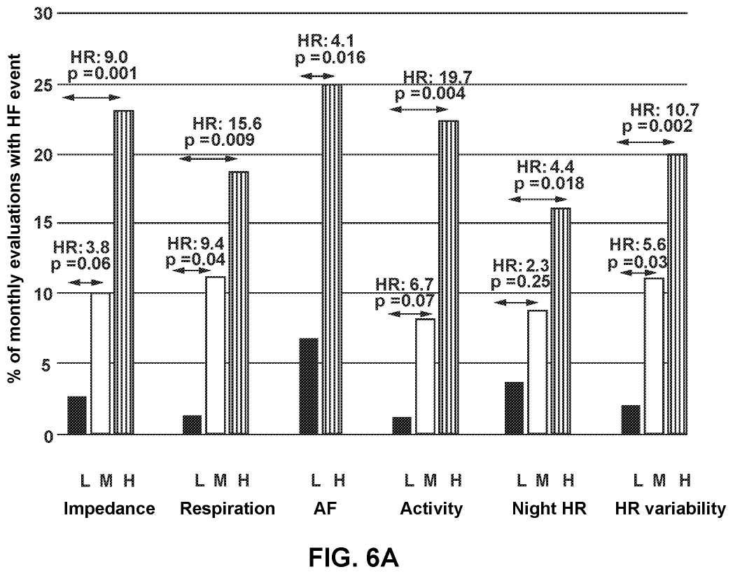

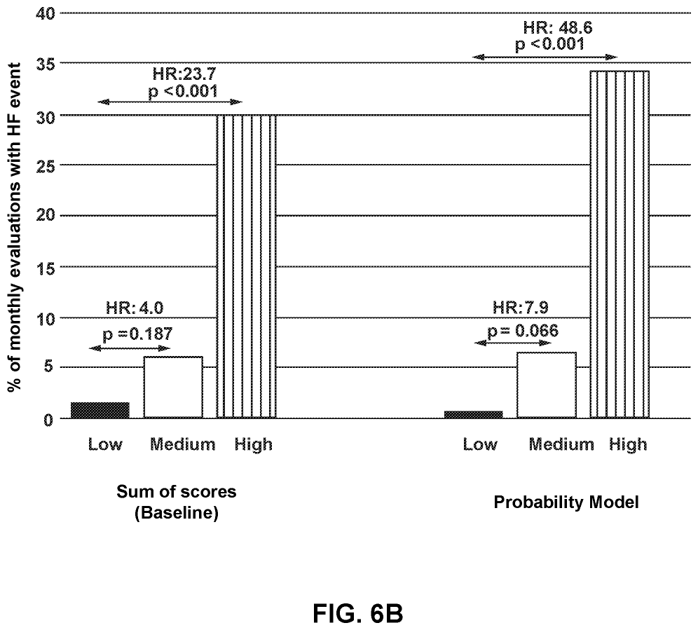

FIG. 6A illustrates a chart of individual parameters that may serve as evidence nodes to the example probability model described with reference to FIG. 4. Each parameter is independent of one another meaning processing circuitry 98 could identify from each individual parameter a risk level for patient 4. However, the accuracy would be less than when using probability model 19 as illustrated in FIG. 6B and Table 2 below. The values for each parameter from FIG. 6A are illustrated in Table 1 below.

TABLE 2

| | # evaluations | #HF hosp (% of | Hazard | prob- |
|---|---|---|---|---|
| | (% of total | evals in each | Ratio | value |
| parameter | of 225 evals) | risk group) | (HR) | (“p-value”) |
| Impedance | | | | 0.006 |
| 0 (L) | 107 (47.6%) | 3 (2.8%) | Reference | |
| 1 (M) | 79 (35.1%) | 8 (10.1%) | 3.84 (0.94-15.62) | 0.061 |
| 2 (H) | 39 (17.3%) | 9 (23.1%) | 9.00 (2.34-34.70) | 0.001 |
| Respiration | | | | 0.029 |
| 0 | 78 (34.7%) | 1 (1.3%) | Reference | |
| 1 | 115 (51.1%) | 13 (11.3%) | 9.39 (1.12-78.60) | 0.039 |
| 2 | 32 (14.2%) | 6 (18.8%) | 15.55 (1.97-122.63) | 0.009 |
| AF | | | | 0.016 |
| 0 | 201 (89.3%) | 14 (7.0%) | Reference | |
| 2 | 24 (10.7%) | 6 (25.0%) | 4.08 (1.30-12.79) | |
| Activity | | | | 0.004 |
| 0 | 79 (35.1%) | 1 (1.3%) | Reference | |
| 1 | 97 (43.1%) | 8 (8.2%) | 6.74 (0.87-52.03) | 0.067 |
| 2 | 49 (21.8%) | 11 (22.4%) | 19.65 (2.55-151.46) | 0.004 |
| NHR | | | | 0.057 |
| 0 | 78 (34.7%) | 3 (3.8%) | Reference | |
| 1 | 91 (40.4%) | 8 (8.8%) | 2.33 (0.55-9.78) | 0.248 |
| 2 | 56 (24.9%) | 9 (16.1%) | 4.40 (1.29-15.02) | 0.018 |
| HRV | | | | 0.010 |
| 0 | 96 (42.7%) | 2 (2.1%) | Reference | |
| 1 | 89 (39.6%) | 10 (11.2%) | 5.59 (1.23-25.37) | 0.026 |
| 2 | 40 (17.8%) | 8 (20.0%) | 10.67 (2.32-49.06) | 0.002 |

FIG. 6B illustrates a sum of individual probability scores from FIG. 6A on the left and a chart of combined probability scores using a probability model based on various evidence nodes of FIG. 4 on the right. For example, FIG. 6B illustrates the advantage of using probability model 19 to model the risk stratification performance for an integrated set of diagnostic states. In the example of FIG. 6B, a Bayesian approach was used to model the probability score shown on the right and an unweighted sum of individual diagnostic states (e.g., PARTNERS-HF or sum of scores) was used on the left. In a non-limiting example, sample results may be summarized in a Table 3 as follows showing a Bayesian approach in comparison to a “sum of scores” approach.

TABLE 3

| | | #HF hosp | Hazard | prob- |
|---|---|---|---|---|
| | | (% of | Ratio | value |
| | # evaluations | evals) | (HR) | (“p-value”) |
| Sum of scores | | | | <0.001 |
| 0-4 | 137 (60.9%) | 2 (1.5%) | Reference | |
| 5 | 34 (15.1%) | 2 (5.9%) | 4.01 (0.51-31.59 | 0.187 |
| 6-12 | 54 (24.0%) | 16 (29.6%) | 23.72 (5.15-109.24) | <0.001 |
| Posterior | | | | <0.001 |
| probability | | | | |
| <1.1% | 119 (59.9%) | 1 (0.8%) | Reference | |
| 1.1%-10% | 62 (27.6%) | 4 (6.5%) | 7.86 (0.87-70.99) | 0.066 |
| >10% | 44 (19.6%) | 15 (34.1%) | 48.61 (6.5-363.51) | <0.001 |

Due to the dynamic nature of the probability score the same patient may have very high evidence at one follow-up and low evidence at another follow-up. The high diagnostic evidence group consisted of evaluations with around the top 20% of risk scores. The combined low evidence group consisted of cases where each or multiple of the diagnostic parameters had low evidence. The results indicate that a patient with a high diagnostic evidence score at a follow-up is 48 times more likely to have an HF event in the next 30 days compared to patients with a low diagnostic evidence score at a follow-up.

FIG. 7 illustrates the environment of an example medical system 2 in conjunction with a patient 4, in accordance with one or more techniques of this disclosure. Patient 4 ordinarily, but not necessarily, will be a human. For example, patient 4 may be an animal needing ongoing monitoring for cardiac conditions.

In some examples, system 2 may include IMD 10. In other examples, system 2 may not include IMD 10 and may instead include other medical device(s) 17 (not shown in FIG. 7). IMD 10 may include one or more electrodes (not shown) on its housing, or may be coupled to one or more leads that carry one or more electrodes. System 2 may also include external device 12 and, although not depicted in FIG. 7, the various other devices illustrated in one or more of the various example techniques described with reference to FIG. 2. Example system 2 may be used to measure subcutaneous impedance to provide to patient 4 other users an early warning for the onset of a heart failure decompensation event.

The example techniques may be used with an IMD 10, which may be in wireless communication with at least one of external device 12 or data server(s) 94. In some examples, IMD 10 is implanted outside of a thoracic cavity of patient 4 (e.g., subcutaneously in the pectoral location illustrated in FIG. 7). IMD 10 may be positioned near the sternum near or just below the level of the heart of patient 4, e.g., at least partially within the cardiac silhouette. IMD 10 may include a plurality of electrodes and may be configured for subcutaneous implantation outside of a thorax of patient 4.

Accordingly, impedance measurements taken via electrodes in the subcutaneous space, e.g., electrodes on a subcutaneously implanted medical device as shown in FIGS. 7-9, may be measurements of the impedance of interstitial fluid and subcutaneous tissue. In an example, during a heart failure decompensation event, reduction in cardiac output can tend to increase venous pressure. An increase in venous pressure tends to lead to an increase in pressure with respect to capillaries compared to the interstitial space. The combination of such tendencies may then lead to a net outflow of fluid from the capillaries into the interstitium or interstitial space of a patient. In such instances, the interstitium will have an increase in fluid accumulation. An increase in fluid accumulation tends to provide a reduction in impedance measured between electrodes.

Implantable medical devices (IMDs) can sense and monitor impedance signals and use those signals to determine a health condition status of a patient or other health condition status of a patient (e.g., edema, preeclampsia, hypertension, etc.). The electrodes used by IMDs to sense impedance signals are typically integrated with a housing of the IMD and/or coupled to the IMD via one or more elongated leads. Example IMDs that include electrodes include the Reveal LINQ™ Insertable Cardiac Monitor (ICM), developed by Medtronic, Inc., of Minneapolis, MN, which may be inserted subcutaneously. Other example IMDs may include electrodes on a subcutaneous lead connected to another one of medical device(s) 17, such as a subcutaneous implantable cardioverter-defibrillator (ICD) or an extravascular ICD. Such IMDs may facilitate relatively longer-term monitoring of patients during normal daily activities and may periodically transmit collected data to a network service, such as the Medtronic CareLink® Network.

Medical devices configured to measure impedance via implanted electrodes, including the examples identified herein, may implement the techniques of this disclosure for measuring impedance changes in the interstitial fluid of a patient to determine whether the patient is experiencing worsening heart failure or decompensation. The techniques include evaluation of the impedance values using criteria configured to provide a desired sensitivity and specificity of heart failure detection. The techniques of this disclosure for identifying heart failure worsening may facilitate determinations of cardiac wellness and risk of sudden cardiac death and may lead to clinical interventions to suppress heart failure worsening, such as with medications.

As such, IMD 10 may be configured to measure, in some cases among other physiological parameter values, impedance values within the interstitial fluid of patient 4. For example, IMD 10 may be configured to receive one or more signals indicative of subcutaneous tissue impedance from electrodes 16. In some examples, IMD 10 may be a purely diagnostic device. For example, IMD 10 may be a device that only determines subcutaneous impedance parameters of patient 4, or a device that determines subcutaneous impedance parameters as well as other physiological parameter values of patient 4. IMD 10 may use the impedance value measurements to determine one or more fluid index values, impedance scores, and/or various thresholds, such as adaptive thresholds, scoring thresholds, weighting factors for thresholds, and/or cardiac risk thresholds.

Subcutaneous impedance may be measured by delivering a signal through an electrical path between electrodes. In some examples, the housing of IMD 10 may be used as an electrode in combination with electrodes located on leads. For example, system 2 may measure subcutaneous impedance by creating an electrical path between a lead and one of the electrodes. In additional examples, system 2 may include an additional lead or lead segment having one or more electrodes positioned subcutaneously or within the subcutaneous layer for measuring subcutaneous impedance. In some examples, two or more electrodes useable for measuring subcutaneous impedance may be formed on or integral with the housing of IMD 10.

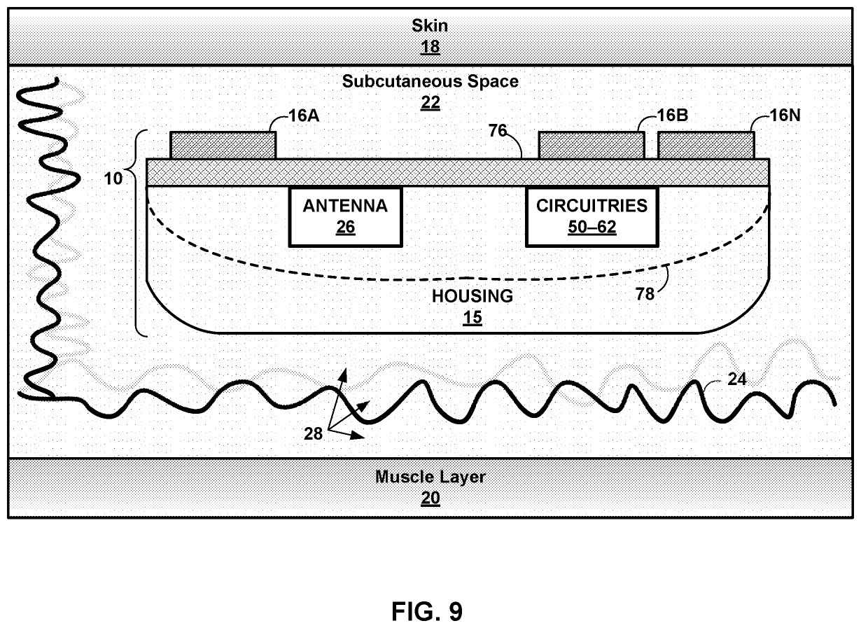

System 2 measures subcutaneous impedance of patient 4 and processes impedance data to accumulate evidence of decreasing impedance. The accumulated evidence is referred to as a fluid index and may be determined as function of the difference between measured impedance values and reference impedance values. The fluid index may then be used to determine impedance scores that are indicative of a heart condition of patient 4. In an example, an impedance score may be measured against a risk threshold that identifies diagnostic states of the subcutaneous tissue impedance physiological parameters, which may be applied to probability model 19 as described herein. In some examples, subcutaneous impedance may provide information about fluid volume in the subcutaneous space (e.g., subcutaneous space 22 of FIG. 9), and in some instances, total blood volume, as well. In such examples, subcutaneous impedance measurements allow system 2 via probably model 19 to identify patients that have accumulated threshold levels of peripheral fluid as determined based on a plurality of evidence nodes, where at least one evidence node is based at least in part on a subcutaneous impedance measurement or subcutaneous impedance score.

In some examples, IMD 10 may also sense cardiac electrogram (EGM) signals via the plurality of electrodes and/or operate as a therapy delivery device. For example, IMD 10 may additionally operate as a therapy delivery device to deliver electrical signals to the heart of patient 4, such as an implantable pacemaker, a cardioverter, and/or defibrillator, a drug delivery device that delivers therapeutic substances to patient 4 via one or more catheters, or as a combination therapy device that delivers both electrical signals and therapeutic substances.

In some examples, system 2 may include any suitable number of leads coupled to IMD 10, and each of the leads may extend to any location within or proximate to a heart or in the chest of patient 4. For example, other examples therapy systems may include three transvenous leads and an additional lead located within or proximate to a left atrium of a heart. As other examples, a therapy system may include a single lead that extends from IMD 10 into a right atrium or right ventricle, or two leads that extend into a respective one of a right ventricle and a right atrium.

In some examples, IMD 10 may be implanted subcutaneously in patient 4. Furthermore, in some examples, external device 12 may monitor subcutaneous impedance values. In some examples, IMD 10 takes the form of the Reveal LINQ™ ICM, or another ICM similar to, e.g., a version or modification of, the LINQ™ ICM, which may be inserted subcutaneously. Such IMDs may facilitate relatively longer-term monitoring of patients during normal daily activities, and may periodically transmit collected data to a network service, such as the Medtronic CareLink® Network.

Although described in the context of examples in which IMD 10 includes an insertable or implantable IMD, example systems including one or more external devices of any type configured to sense subcutaneous tissue impedances may be configured to implement the techniques of this disclosure. In some examples, IMD 10 may be a device configured to measure impedances of a fluid and shifts in impedances of the fluid, such as interstitial fluid. For example, IMD 10 may have one or more electrodes disposed within one layer of patient 4 (e.g., subcutaneous layer), whereas at least one other electrode may be disposed within another layer of patient 4 (e.g., dermis layer, muscle layer, etc.). In such examples, IMD 10 may be able to measure impedances and shifts in impedances of the interstitial fluid of the subcutaneous layer. In another example, IMD 10 may be a cutaneous patch device having electrodes on the outside of the skin. In such examples, IMD 10 may use the cutaneous patch device to measure impedances and shifts in impedances of the interstitial fluid in the subcutaneous layer.

In examples in which IMD 10 also operates as a pacemaker, a cardioverter, and/or defibrillator, or otherwise monitors the electrical activity of the heart, IMD 10 may sense electrical signals attendant to the depolarization and repolarization of the heart of patient 4 via electrodes of or coupled to IMD 10, e.g., which may include the electrodes used to determine subcutaneous impedance. In some examples, IMD 10 can provide pacing pulses to the heart of patient 4 based on the electrical signals sensed within the heart of patient 4. The configurations of electrodes used by IMD 10 for sensing and pacing may be unipolar or bipolar. IMD 10 may also provide defibrillation therapy and/or cardioversion therapy via electrodes located on at least one lead, as well as a housing electrode. IMD 10 may detect tachyarrhythmia of the heart of patient 4, such as fibrillation of atria or ventricles, and deliver defibrillation or other tachyarrhythmia therapy to the heart of patient 4 in the form of electrical pulses. In some examples, IMD 10 may be programmed to deliver a progression of therapies, e.g., pulses with increasing energy levels, until a fibrillation of the heart of patient 4 is stopped. IMD 10 detects fibrillation or other tachyarrhythmias employing tachyarrhythmia detection techniques known in the art.

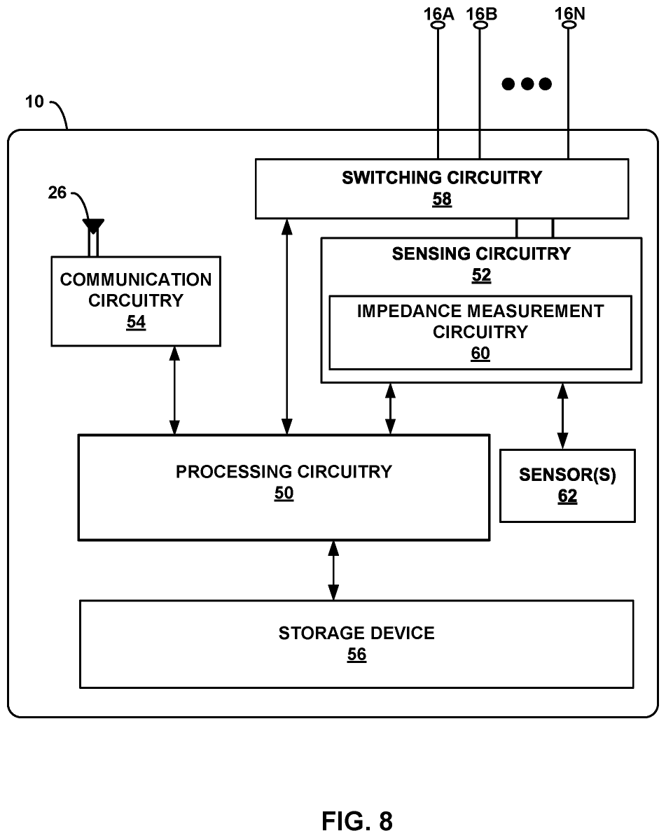

FIG. 8 is a functional block diagram illustrating an example configuration of IMD 10. IMD 10 may include an example of one of medical device(s) 17 described with reference to FIGS. 2-4. In the illustrated example, IMD 10 includes electrodes 16A-16N (collectively, “electrodes 16”), antenna 26, processing circuitry 50, sensing circuitry 52, impedance measurement circuitry 60, communication circuitry 54, storage device 56, switching circuitry 58, sensors 62. IMD 10, along with other medical device(s) 17, may also include a power source. In general, the power source may include a rechargeable or non-rechargeable battery. Each of medical device(s) 17 may include components common to those of IMD 10. For example, each of medical device(s) 17 may include processing circuitry 50. For sake of brevity, each configuration of each medical device(s) 17 will not be described in this application. That is, certain components of IMD 10 may serve as representative components of other medical device(s) 17 (e.g., storage device 56, communication circuitry 54, sensor(s) 62, etc.).

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to select the electrodes 16 and polarity, referred to as the sensing vector, used to sense impedance and/or cardiac signals, as controlled by processing circuitry 50. Sensing circuitry 52 may sense signals from electrodes 16, e.g., to produce a cardiac EGM, in order to facilitate monitoring the electrical activity of the heart. Sensing circuitry 52 also may monitor signals from sensors 62, which may include one or more sensors configured to detect any of the physiological parameters described herein, such as one or more accelerometers, pressure sensors, temperature sensors, glucose or other analyte sensors, optical sensors, as examples. In some examples, sensing circuitry 52 may include one or more filters and amplifiers for filtering and amplifying signals received from electrodes 16 and/or sensors 62.

In some examples, processing circuitry 50 may use switching circuitry 58 to select, e.g., via a data/address bus, which of the available electrodes are to be used to obtain impedance measurements of interstitial fluid and to sense cardiac signals, and to select the polarities of the electrodes. Switching circuitry 58 may include a switch array, switch matrix, multiplexer, transistor array, microelectromechanical switches, or any other type of switching device suitable to selectively couple sensing circuitry 58 to selected electrodes. In some examples, sensing circuitry 52 includes one or more sensing channels, each of which may include an amplifier. In response to the signals from processing circuitry 50, switching circuitry 58 may couple the outputs from the selected electrodes to one of the sensing channels.

In some examples, one or more channels of sensing circuitry 52 may include one or more R-wave amplifiers that receive signals from electrodes 16. In some examples, the R-wave amplifiers may take the form of an automatic gain-controlled amplifier that provides an adjustable sensing threshold as a function of the measured R-wave amplitude. In addition, in some examples, one or more channels of sensing circuitry 52 may include a P-wave amplifier that receives signals from electrodes 16. Sensing circuitry may use the received signals for pacing and sensing in the heart of patient 4. In some examples, the P-wave amplifier may take the form of an automatic gain-controlled amplifier that provides an adjustable sensing threshold as a function of the measured P-wave amplitude. Other amplifiers may also be used.

In some examples, processing circuitry 50 may be configured to record an R-wave amplitude for an ECG sensed by sensing circuitry 52. For example, sensing circuitry 52 may be configured to sense a subcutaneous ECG, and processing circuitry 50 may be configured to record an R-wave amplitude of the subcutaneous ECG. In another example, sensing circuitry 52 may be configured to record cardiac electrogram using leads in the heart of patient 4 and as measured between the housing of one of medical device(s) 17 (e.g., a can) and the leads in the heart of patient 4, and processing circuitry 50 may be configured to record an R-wave amplitude of the cardiac electrogram. Similarly, sensing processing circuitry 50 may record a R-wave slopes or R-wave widths for an ECG or other cardiac electrogram.

In some examples, sensing circuitry 52 includes a channel that includes an amplifier with a relatively wider pass band than the R-wave or P-wave amplifiers. Signals from the selected sensing electrodes that are selected for coupling to this wide-band amplifier may be provided to a multiplexer, and thereafter converted to multi-bit digital signals by an analog-to-digital converter for storage in storage device 56. In some examples, processing circuitry 50 may employ digital signal analysis techniques to characterize the digitized signals stored in storage device 56 to detect P-waves (e.g., within ventricular or far-field signals and instead of or in addition to use of P-wave amplifiers) and classify cardiac tachyarrhythmias from the digitized electrical signals.

Based on the detection R-waves and P-waves, e.g., their rates, processing circuitry 50 may identify atrial and ventricular tachyarrhythmias, such as AF or VF. Processing circuitry may employ digital signal analysis techniques to detect or confirm such tachyarrhythmias in some examples. Processing circuitry 50 may determine values of parameters based on detection of such tachyarrhythmias, and a probability of a health event may be determined based on the parameter values according to the techniques described herein. Example parameters determined based on detection of tachyarrhythmia include an extent, e.g., frequency and/or duration during a time period, of AF or other tachyarrhythmias.

Processing circuitry 50 may also determine other parameter values that can be used to determine probability of a health event based on the cardiac EGM and detection of depolarizations therein. As examples, processing circuitry 50 may determine one or more heart rate values, such as night heart rate values, one or more heart rate variability values. As other examples, processing circuitry 50 may determine magnitudes of or intervals between features within the cardiac EGM, such as depolarization amplitudes, depolarization widths, or intervals between depolarizations and repolarizations.

In some examples, sensors 62 include one or more accelerometers or other sensors configured to generate signals that indicate motion and orientation of patient 4, e.g., that indicate activity level or posture of the patient. In some examples, processing circuitry 50 processes such signals to determine values of one or more parameters that can be used to determine probability of a health event. For example, processing circuitry 50 may quantify duration, frequency, and/or intensity of activity and/or posture changes, e.g., daily or during some other period. In some examples, processing circuitry 50 may determine an amount of time patient spends inactive, e.g., sleeping, but not in a supine posture based on such signals.

Sensing circuitry 52 includes impedance measurement circuitry 60. Processing circuitry 50 may control impedance circuitry 60 to periodically measure an electrical parameter to determine an impedance, such as a subcutaneous impedance indicative of fluid found in interstitium 28. For a subcutaneous impedance measurement, processing circuitry 50 may control impedance measurement circuitry 60 to deliver an electrical signal between selected electrodes 16 and measure a current or voltage amplitude of the signal. Processing circuitry 50 may select any combination of electrodes 16, e.g., by using switching circuitry 58 and sensing circuitry 52. Impedance measurement circuitry 60 includes sample and hold circuitry or other suitable circuitry for measuring resulting current and/or voltage amplitudes. Processing circuitry 50 determines an impedance value from the amplitude value(s) received from impedance measurement circuitry 60.

Because either IMD 10 or external device 12 may be configured to include sensing circuitry 52, impedance measurement circuitry 60 may be implemented in one or more processors, such as processing circuitry 50 of IMD 10 or processing circuitry 80 of external device 12. Impedance measurement circuitry 60 is, in this example, shown in conjunction with sensing circuitry 52 of IMD 10. Impedance measurement circuitry 60 may be embodied as one or more hardware modules, software modules, firmware modules, or any combination thereof. Impedance measurement circuitry 60 may analyze impedance measurement data on a periodic basis to identify a decrease in subcutaneous impedance in patient 4 and alert patient 4 when the decrease indicates onset of a possible heart failure decompensation event.

In some examples, processing circuitry 50 may perform an impedance measurement by causing impedance measurement circuitry 60 (via switching circuitry 58) to deliver a voltage pulse between at least two electrodes 16 and examining resulting current amplitude value measured by impedance measurement circuitry 60. In some examples, switching circuitry 58 delivers signals that do deliver stimulation therapy to the heart of patient 4. In other examples, these signals may be delivered during a refractory period, in which case they may not stimulate the heart of patient 4.

In some examples, processing circuitry 50 may perform an impedance measurement by causing impedance measurement circuitry 60 (via switching circuitry 58) to deliver a current pulse across at least two selected electrodes 16. Impedance measurement circuitry 60 holds a measured voltage amplitude value. Processing circuitry 50 determines an impedance value based upon the amplitude of the current pulse and the amplitude of the resulting voltage that is measured by impedance measurement circuitry 60. IMD 10 may use defined or predetermined pulse amplitudes, widths, frequencies, or electrode polarities for the pulses delivered for these various impedance measurements. In some examples, the amplitudes and/or widths of the pulses may be sub-threshold, e.g., below a threshold necessary to capture or otherwise activate tissue, such as cardiac tissue, subcutaneous tissue, or muscle tissue. In some examples, IMD 10 may use an amplifier circuit to perform physiological signal sensing, impedance sensing, telemetry, etc.

In certain cases, IMD 10 may measure subcutaneous impedance values that include both a resistive component and a reactive component (e.g., X, XL, XC), such as in an impedance triangle. In such cases, IMD 10 may measure subcutaneous impedance during delivery of a sinusoidal or other time varying signal by impedance measurement circuitry 60, for example. Thus, as used herein, the term “impedance” is used in a broad sense to indicate any collected, measured, and/or calculated value that may include one or both of resistive and reactive components. In some examples, subcutaneous tissue impedance parameters are derived from subcutaneous tissue impedance signals received from electrodes 16.

Sensing circuitry 52 may also provide one or more impedance signals to processing circuitry 50 for analysis, e.g., for analysis to determine respiration and impedance parameters, e.g., impedance scores. In some examples, processing circuitry 50 may store the impedance values, impedance score factors (e.g., fluid indices, average impedance values, reference impedance values, buffer values, etc.), and impedance scores in storage device 56. Processing circuitry 50 of IMD 10, and/or processing circuitry of another device that retrieves data from IMD 10, may analyze the impedance values to determine a diagnostic state of the subcutaneous tissue impedance parameter.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network.

Antenna 26 and communication circuitry 54 may be configured to transmit and/or receive signals via inductive coupling, electromagnetic coupling, NFC technologies, RF communication, Bluetooth®, Wi-Fi™, or other proprietary or non-proprietary wireless communication schemes. In some examples, processing circuitry 50 may provide data to be uplinked to external device 12 via communication circuitry 54 and control signals using an address/data bus. In another example, communication circuitry 54 may provide received data to processing circuitry 50 via a multiplexer.

In some examples, processing circuitry 50 may send impedance data to external device 12 or data server(s) 94 via communication circuitry 54. For example, IMD 10 may send external device 12 or data server(s) 94 collected impedance measurements. External device 12 and/or data server(s) 94 may then analyze those impedance measurements.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media. For example, storage device 56 may include random access memory (RAM), read-only memory (ROM), non-volatile RAM (NVRAM), electrically-erasable programmable ROM (EEPROM), erasable programmable ROM (EPROM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include impedance values and/or digitized cardiac EGMs, as examples.

In some examples, processing circuitry, e.g., processing circuitry 50 of IMD 10, processing circuitry 80 of external device 12, or processing circuitry 98 of data server(s) 94, may determine RRs or other respiration parameters based on analysis of impedance values determined as described herein but, in some cases, sampled at a higher rate than for detecting changes in the fluid status of patient 4. For example, processing circuitry 50 (or processing circuitry of another device) may employ any of a variety of techniques to detect the frequency, period between, or magnitude of fluctuations in the impedance values associated with respiration of patient 4. In some examples, processing circuitry 50 may control impedance measurements for determining respiration parameters to occur when certain conditions are satisfied, e.g., time of day, such as night, or patient activity level or posture.

In some examples, processing circuitry, e.g., processing circuitry 50 of IMD 10, processing circuitry 80 of external device 12, or processing circuitry 98 of data server(s) 94, may determine an impedance score based on triggering events that indicate a subcutaneous tissue impedance event of patient 4. For example, processing circuitry 98 may increment the impedance score by a first value in response to a first group of one or more triggering events. In some examples, processing circuitry 98 may increment the impedance score by a second value in response to a second group of one or more triggering events. The first value, in some examples, may increment the impedance score by one point, whereas the second value, in some examples, may increment the impedance score by two points. Other point values may be used that are greater than or less than the first value or the second value. In some examples, processing circuitry 98 may detect multiple triggering events during a single iteration of the scoring cycle, in which case a sum of values may be applied to the impedance score. For example, processing circuitry 98 may increment the impedance score by four points when processing circuitry 98 determines that two triggering events are present, one triggering event corresponding to a two-point incremental value and where another triggering event also corresponds to a two-point incremental value. In some examples, processing circuitry 98 may determine a total impedance score between a value of 0 on the low end and 7 on the high end, as discussed herein.

In some examples, processing circuitry, e.g., processing circuitry 50 of IMD 10, processing circuitry 80 of external device 12, or processing circuitry 98 of data server(s) 94, may determine a diagnostic state based on the impedance score. For example, processing circuitry 98 may periodically compare the impedance score to one or more risk thresholds. For example, processing circuitry 98 may perform a comparison of the impedance score to the risk thresholds at a same time each day (e.g., at the end of the day). In some examples, processing circuitry 98 may determine the diagnostic state at multiple intervals each day. In yet another example, processing circuitry 98 may determine the diagnostic state at longer intervals, such as once a week or once every two weeks.

In some examples, processing circuitry 98 may determine a diagnostic state as a heart failure risk status. The risk status may be determined as low, medium, high, etc. In some examples, processing circuitry 98 may use a different number of risk categories, such as including a category for very high risk in some instances or very low risk. In addition, processing circuitry 98 may not include certain categories, such as the medium risk category, and instead only monitor low and high-risk categories. In some examples, processing circuitry 98 may compare the impedance score to risk thresholds to determine a diagnostic state for the subcutaneous tissue impedance parameter.

In a non-limiting example, processing circuitry 98 may determine diagnostic states for the subcutaneous tissue impedance parameter as follows: low risk if the impedance score is 0, medium risk if the impedance score is greater than or equal to 1 but less than or equal to 6, and high if the impedance score is greater than or equal to 7. Risk thresholds may be set (e.g., programmably by a user) based on optimization considerations and may be based on the specific values used to determine fluid index values.

In some examples, processing circuitry, e.g., processing circuitry 50 of IMD 10, processing circuitry 80 of external device 12, or processing circuitry 98 of data server(s) 94, may determine satisfaction of at least one of: a scoring threshold and an impedance threshold, with respect to one or more time windows. For example, processing circuitry 98 modify the impedance score in response to the one or more fluid index values satisfying one or more scoring thresholds for at least one of: a predetermined amount of time and a predetermined number of times (e.g., number of days, etc.). In an illustrative example, processing circuitry 98 may increment the impedance score by a point value (e.g., a 1 point value) in response to the following example conditions (e.g., scoring thresholds) being satisfied with respect to the first time period: (1) the fluid index values in the last 30 days have been greater than or equal to the adaptive threshold (multiplied by 0.6) for one or more days; (2) the fluid index values in the last 30 days have been greater than or equal to the adaptive threshold (multiplied by 1.7) for one or more days; or (3) the fluid index values in the last 30 days have been greater than or equal to the adaptive threshold (multiplied by 3.2) for one or more days. In this example, processing circuitry 98 determined the weighting factors as 0.6, 1.7, and 3.2. In this example, the first time period is the last 30 days. However, as discussed herein, the time periods and the weighting factors may vary depending on specifics related to patient 4, for example.

In another example, processing circuitry 98 may also increment the impedance score by a point value of greater than one (e.g., two points) in response to the average impedance satisfying an impedance value threshold and the fluid index satisfying scoring thresholds. In some examples, processing circuitry 98 may modify the impedance score in response to the average impedance value satisfying an impedance value threshold. The impedance value threshold may, in some examples, be less than or equal to approximately 600 ohms or another comparable ohm value.

For example, processing circuitry 98 may increment the impedance score by two points in response to the following example conditions (e.g., scoring thresholds and impedance value thresholds) being met with respect to the first time period: (1) the fluid index values in the last 30 days have been greater than or equal to the adaptive threshold (multiplied by 1.5) for 24 or more days; or (2) the average impedance in the last 30 days has been less than or equal to approximately 600 ohms. For the first condition, the 24 or more days may be consecutive days or instead may be a cumulative 24 days. For the average impedance, the average impedance in the last 30 days may refer to a set of daily average impedances in the last 30 days. In some examples, the average impedance in the last 30 days may refer to a single average of the impedance values measured over time. In another example, the average impedance may refer to a single average of the daily average impedance values determined over time.

In some examples, processing circuitry 98 may determine anew or modify an impedance score when the fluid index values during the second time period satisfy the adaptive threshold multiplied by the corresponding weighting factors. In addition, processing circuitry 98 may determine anew or modify an impedance score when the average impedance satisfies an impedance threshold during the second time period.

In an illustrative example, processing circuitry 98 may increment the impedance score by a point value equal to one in response to the following example conditions being satisfied with respect to the second time period: (1) the fluid index values in the last seven days have been greater than or equal to the adaptive threshold (multiplied by 0.6) for one or more days; (2) the fluid index values in the last seven days have been greater than or equal to the adaptive threshold (multiplied by 1.7) for one or more days; or (3) the fluid index values in the last seven days have been greater than or equal to the adaptive threshold (multiplied by 1.5) for seven or more days. In this example, IMD 10 determined the weighting factors as 0.6, 1.7, and 1.5. In this example, the second time period is the last seven days. However, as discussed herein, the time periods and the weighting factors may vary depending on specifics related to patient 4, for example. In addition, for the last condition, the 7 or more days may be consecutive days or instead may be a cumulative 7 days.

In another example, IMD 10 may also increment the impedance score by a point value of greater than one (e.g., two points) in response to other example conditions being met with respect to the second time period: (1) the fluid index values in the last seven days have been greater than or equal to the adaptive threshold (multiplied by 3.2) for one or more days; or (2) the average impedance in the last seven days has been less than or equal to approximately 600 ohms. For the average impedance, the average impedance in the last 7 days may refer to a set of daily average impedances in the last 7 days. In some examples, the average impedance in the last 7 days may refer to a single average of the impedance values measured over time. In another example, the average impedance may refer to a single average of the daily average impedance values determined over time.

In some examples, where overlaps exist between conditions, only the higher point value would be added to the impedance score so as to avoid any compounding affects to the modification of the impedance score. In keeping with the example described above, where two conditions are met (e.g., average impedance in last 7 days and in the last 30 days has been greater than or equal to approximately 600 ohms), the impedance score may only increment by two and not by four. In other examples, where two conditions are met (e.g., average impedance in last 7 days and in the last 30 days has been greater than or equal to approximately 600 ohms), IMD 10 may increment the impedance score based on both conditions being satisfied. The impedance score may be used to determine a diagnostic state of the subcutaneous tissue impedance physiological parameter to serve as one of evidence nodes 8.

As noted before with reference to FIGS. 4 and 5, the above techniques of determining an impedance score may also be performed on a periodic basis. For example, the impedance scores may be determined according to a resolution parameter setting of processing circuitry 50 (e.g., the resolution parameter used to signal a frequency at which electrodes 16 should probe for impedance measurements). In other examples, the impedance score may be calculated irrespective of the resolution parameter, which, for example, may apply to the fluid index determination and/or the reference impedance value determination, but not the impedance score determination. For instance, processing circuitry 50 may calculate the impedance scores at several time intervals each day (e.g., once in the morning, once in the afternoon, once in the evening, once after meals, etc.). In some examples, processing circuitry 50 may calculate the impedance score once a day, each week, every two weeks, each month, etc. In some examples, processing circuitry 50 may also calculate the impedance score in response to a user command (e.g., from a physician, from a user interface) or in response to a satisfaction of another condition (e.g., based on activity level or other physiological parameters). For example, processing circuitry 50 may determine impedance score on a per measurement basis, such as on a per fluid index determination basis or on a per impedance measurement basis. A person of skill in the art should appreciate that various periods may exist for when IMD 10, data server(s) 94, or external device 12 may transmit impedance scores, receive impedance scores, receive fluid index values, and/or otherwise, calculate impedance scores for subsequent analysis.

FIG. 9 is a conceptual side-view diagram illustrating an example configuration of an IMD, such as IMD 10 described with reference to FIGS. 7 and 8. The conceptual side-view diagram illustrates a muscle layer 20 and a skin layer 18 (e.g., dermis layer, epidermis layer). The region between muscle layer 20 and skin layer 18 includes subcutaneous space 22. Subcutaneous space includes blood vessels 24, such as capillaries, arteries, or veins, and interstitial fluid in the interstitium 28 of subcutaneous space 22. Subcutaneous space 22 has interstitial fluid that is commonly found between skin 18 and muscle layer 20. Subcutaneous space 22 may include interstitial fluid that surrounds blood vessels 24. For example, interstitial fluid surrounds capillaries and allows the passing of capillary elements (e.g., nutrients) between the different layers of a body through interstitium 28.

In the example shown in FIG. 9, IMD 10 may include a leadless, subcutaneously implantable monitoring device having a housing 15 and an insulative cover 76. Electrodes 16 may be formed or placed on an outer surface of cover 76. Although the illustrated example includes three electrodes 16, IMDs including or coupled to more or less than three electrodes 16 may implement the techniques of this disclosure in some examples. In some examples, electrodes 16 may be disposed all within a single layer, such as subcutaneous space 22 and contact interstitial fluid in subcutaneous space 22.

Circuitries 50-62 may be formed or placed on an inner surface of cover 76, or within housing 15. In the illustrated example, antenna 26 is formed or placed on the inner surface of cover 76, but may be formed or placed on the outer surface in some examples. In some examples, one or more of sensors 62 may be formed or placed on the outer surface of cover 76. In some examples, insulative cover 76 may be positioned over an open housing 15 such that housing 15 and cover 76 enclose antenna 26 and circuitries 50-62, and protect antenna 26 and circuitries from fluids such as interstitial fluids or other bodily fluids.

One or more of antenna 26 or circuitries 50-62 may be formed on the inner side of insulative cover 76, such as by using flip-chip technology. Insulative cover 76 may be flipped onto a housing 15. When flipped and placed onto housing 15, the components of IMD 10 formed on the inner side of insulative cover 76 may be positioned in a gap 78 defined by housing 15. Electrodes 16 may be electrically connected to switching circuitry 58 through one or more vias (not shown) formed through insulative cover 76. Insulative cover 76 may be formed of sapphire (i.e., corundum), glass, parylene, and/or any other suitable insulating material. Housing 15 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

FIG. 10 is a flow diagram illustrating an example process that may be performed by one or more medical devices and/or processing devices, in accordance with one or more techniques disclosed herein. The processing device may correspond to any one or more of external device 12, data server(s) 94, computing devices 100, or other such processing devices. The medical devices may correspond to any of IMD 10, medical device(s) 17, or other such medical devices. In some cases, medical devices may also be processing devices, and vice versa.

The processing device receives medication data that is indicative of compliance of a patient with a prescribed medication plan (1002). To obtain the medication data for the patient, the processing device may be configured to solicit user input indicating the compliance with the prescribed medication plan or receive automated confirmations that doses of medication have either been taken or not taken. In some implementations, a medical device may automatically detect medication compliance based on a physiological signature of the medication taken. Different drug types typically have different effects on physiology and taking a medication may have a transient effect on certain patient parameters. For example, a beta blocker may have an effect on heart rate, and perhaps cause a transient decrease in HR (or HRV) that resolves over a period of time. A medical device could be configured to detect this change in HR, which can serve as a confirmation that a drug was taken. The medical device can periodically relay such confirmations or absences of confirmations to the processing devices.

There are a number of ways to determine a signature for each drug or a combination of drugs. For example, a medical device could monitor known changes in physiology to predict an individual response to then detect the taking of a drug. The medical device could be trained using ML or other techniques to detect such changes. For a period of time, a patient could indicate via user input when a particular drug was taken, and the medical device could detect changes in parameters (HR, HRV, impedance, vascular tone, BP, EKG changes, etc.) to assign a template response to a particular patient-drug combination. Additionally, a particular medication, or combined formulation of a medication, may have an added compound that induces a distinctive change in physiology. This extra compound may not have an effect on the patient's long-term physiology, but could produce a very short-term identifiable change. This could be in HR, EKG component change, etc. A medical device could detect this distinctive change to sense a medication that was taken.

The processing devices determines a compliance metric for the patient with the prescribed medication plan based on the medication data (1004). The compliance metric is indicative of a degree of compliance of the patient with the prescribed medication plan and may, may, for example, correspond to a diagnostic state selected from a group of diagnostic states. The processing device may be configured to determine the compliance metric based on a frequency with which the patient takes the medication relative to an expected frequency established by the prescribed medication plan, based on a deviation from times in which the patient takes the medication relative to expected times established by the prescribed medication plan, based on an amount of medication taken during a time period relative to an expected amount of medication taken during the time period as established by the prescribed medication plan, based on a number of doses missed within a period of time, or based on any other such criteria.

The processing device obtains, via one or more medical devices, values for one or more physiological parameters for the patient (1006). The one or more physiological parameters may, for example, include a subcutaneous tissue impedance parameter or any other such parameter described herein. The physiological parameters may also correspond to diagnostic states in the manner described above.

The processing device determines a heart failure risk score for the patient based on the compliance metric and the values for the one or more physiological parameters for the patient (1008). To determine the heart failure risk score for the patient, the one or more processors are configured to input the compliance metric and the values for the one or more physiological parameters for the patient into a probability model, such as a Bayesian network or other such model, like the ones described in this disclosure.

The processing device may be configured to generate an alert in response to the heart failure risk score meeting or exceeding a threshold or otherwise in response to determining that a change in the heart failure risk score for the patient correlates to a lack of compliance of the patient with the prescribed medication plan. In some examples, the processing device may additionally or alternatively provide the patient with a recommended course of action to improve their heart failure risk score. The recommended course of action may be reminders to follow the prescribed medication plan and, in some examples, may include modifications to the prescribed medication plan, such as to take a diuretic in response to a medication being missed.

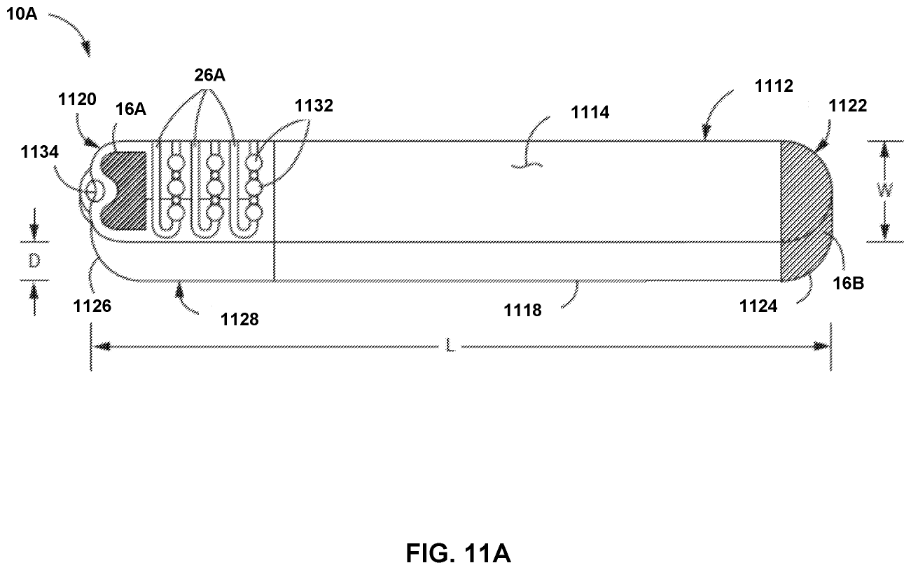

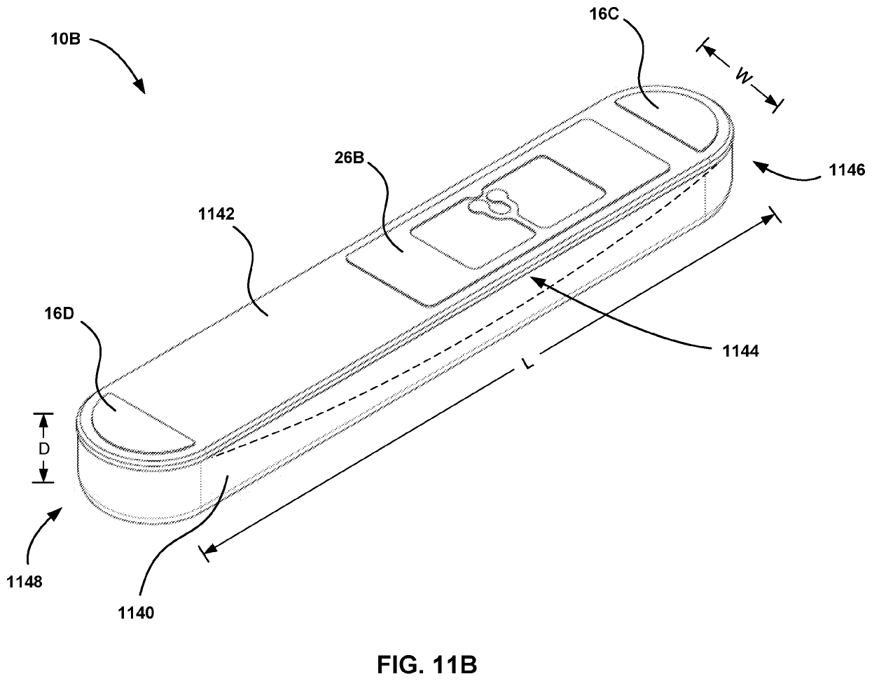

FIG. 11A is a perspective drawing illustrating an IMD 10A, which may be an example configuration of IMD 10 of FIGS. 7-9 as an ICM. In the example shown in FIG. 11A, IMD 10A may be embodied as a monitoring device having housing 1112, proximal electrode 16A and distal electrode 16B. Housing 1112 may further comprise first major surface 1114, second major surface 1118, proximal end 1120, and distal end 1122. Housing 1112 encloses electronic circuitry located inside the IMD 10A and protects the circuitry contained therein from body fluids. Housing 1112 may be hermetically sealed and configured for subcutaneous implantation. Electrical feedthroughs provide electrical connection of electrodes 16A and 16B.

In the example shown in FIG. 11A, IMD 10A is defined by a length L, a width W and thickness or depth D and is in the form of an elongated rectangular prism wherein the length L is much larger than the width W, which in turn is larger than the depth D. In one example, the geometry of the IMD 10A-in particular a width W greater than the depth D-is selected to allow IMD 10A to be inserted under the skin of the patient using a minimally invasive procedure and to remain in the desired orientation during insertion. For example, the device shown in FIG. 11A includes radial asymmetries (notably, the rectangular shape) along the longitudinal axis that maintains the device in the proper orientation following insertion. For example, the spacing between proximal electrode 46A and distal electrode 46B may range from 5 millimeters (mm) to 55 mm, 30 mm to 55 mm, 35 mm to 55 mm, and from 40 mm to 55 mm and may be any range or individual spacing from 5 mm to 60 mm. In addition, IMD 10A may have a length L that ranges from 30 mm to about 70 mm. In other examples, the length L may range from 5 mm to 60 mm, 40 mm to 60 mm, 45 mm to 60 mm and may be any length or range of lengths between about 30 mm and about 70 mm. In addition, the width W of major surface 14 may range from 3 mm to 15, mm, from 3 mm to 10 mm, or from 5 mm to 15 mm, and may be any single or range of widths between 3 mm and 15 mm. The thickness of depth D of IMD 10A may range from 2 mm to 15 mm, from 2 mm to 9 mm, from 2 mm to 5 mm, from 5 mm to 15 mm, and may be any single or range of depths between 2 mm and 15 mm. In addition, IMD 10A according to an example of the present disclosure is has a geometry and size designed for ease of implant and patient comfort. Examples of IMD 10A described in this disclosure may have a volume of three cubic centimeters (cm) or less, 1.5 cubic cm or less or any volume between three and 1.5 cubic centimeters.

In the example shown in FIG. 11A, once inserted within the patient, the first major surface 1114 faces outward, toward the skin of the patient while the second major surface 1118 is located opposite the first major surface 1114. In addition, in the example shown in FIG. 11A, proximal end 1120 and distal end 1122 are rounded to reduce discomfort and irritation to surrounding tissue once inserted under the skin of the patient. IMD 10A, including instrument and method for inserting IMD 10 is described, for example, in U.S. Patent Publication No. 2014/0276928.

Proximal electrode 16A is at or proximate to proximal end 1120, and distal electrode 16B is at or proximate to distal end 1122. Proximal electrode 16A and distal electrode 16B are used to sense cardiac EGM signals, e.g., ECG signals, thoracically outside the ribcage, which may be sub-muscularly or subcutaneously. EGM signals may be stored in a memory of IMD 10A, and data may be transmitted via integrated antenna 26A to another device, which may be another implantable device or an external device, such as external device 12. In some example, electrodes 16A and 16B may additionally or alternatively be used for sensing any bio-potential signal of interest, which may be, for example, an EGM, EEG, EMG, or a nerve signal, or for measuring impedance, from any implanted location. Housing 1112 may house the circuitry of IMD 10 illustrated in FIG. 8.

In the example shown in FIG. 11A, proximal electrode 16A is at or in close proximity to the proximal end 20 and distal electrode 16B is at or in close proximity to distal end 22. In this example, distal electrode 16B is not limited to a flattened, outward facing surface, but may extend from first major surface 1114 around rounded edges 1124 and/or end surface 1126 and onto the second major surface 1118 so that the electrode 16B has a three-dimensional curved configuration. In some examples, electrode 16B is an uninsulated portion of a metallic, e.g., titanium, part of housing 12.

In the example shown in FIG. 11A, proximal electrode 16A is located on first major surface 1114 and is substantially flat, and outward facing. However, in other examples proximal electrode 16A may utilize the three dimensional curved configuration of distal electrode 16B, providing a three dimensional proximal electrode (not shown in this example). Similarly, in other examples distal electrode 16B may utilize a substantially flat, outward facing electrode located on first major surface 1114 similar to that shown with respect to proximal electrode 16A.

The various electrode configurations allow for configurations in which proximal electrode 16A and distal electrode 16B are located on both first major surface 1114 and second major surface 1118. In other configurations, such as that shown in FIG. 11A, only one of proximal electrode 16A and distal electrode 16B is located on both major surfaces 1114 and 1118, and in still other configurations both proximal electrode 16A and distal electrode 16B are located on one of the first major surface 1114 or the second major surface 1118 (e.g., proximal electrode 16A located on first major surface 1114 while distal electrode 16B is located on second major surface 1118). In another example, IMD 10A may include electrodes on both major surface 1114 and 1118 at or near the proximal and distal ends of the device, such that a total of four electrodes are included on IMD 10A. Electrodes 16A and 16B may be formed of a plurality of different types of biocompatible conductive material, e.g. stainless steel, titanium, platinum, iridium, or alloys thereof, and may utilize one or more coatings such as titanium nitride or fractal titanium nitride.

In the example shown in FIG. 11A, proximal end 1120 includes a header assembly 1128 that includes one or more of proximal electrode 16A, integrated antenna 26A, anti-migration projections 1132, and/or suture hole 1134. Integrated antenna 26A is located on the same major surface (i.e., first major surface 1114) as proximal electrode 16A and is also included as part of header assembly 1128. Integrated antenna 26A allows IMD 10A to transmit and/or receive data. In other examples, integrated antenna 26A may be formed on the opposite major surface as proximal electrode 16A, or may be incorporated within the housing 1112 of IMD 10A. In the example shown in FIG. 11A, anti-migration projections 1132 are located adjacent to integrated antenna 26A and protrude away from first major surface 1114 to prevent longitudinal movement of the device. In the example shown in FIG. 11A, anti-migration projections 1132 include a plurality (e.g., nine) small bumps or protrusions extending away from first major surface 1114. As discussed above, in other examples anti-migration projections 1132 may be located on the opposite major surface as proximal electrode 16A and/or integrated antenna 26A. In addition, in the example shown in FIG. 11A, header assembly 1128 includes suture hole 1134, which provides another means of securing IMD 10A to the patient to prevent movement following insertion. In the example shown, suture hole 1134 is located adjacent to proximal electrode 16A. In one example, header assembly 1128 is a molded header assembly made from a polymeric or plastic material, which may be integrated or separable from the main portion of IMD 10A.

FIG. 11B is a perspective drawing illustrating another IMD 10B, which may be another example configuration of IMD 10 from FIGS. 7-9 as an ICM. IMD 10B of FIG. 11B may be configured substantially similarly to IMD 10A of FIG. 11A, with differences between them discussed herein.

IMD 10B may include a leadless, subcutaneously-implantable monitoring device, e.g. an ICM. IMD 10B includes housing having a base 1140 and an insulative cover 1142. Proximal electrode 16C and distal electrode 16D may be formed or placed on an outer surface of cover 1142. Various circuitries and components of IMD 10B, e.g., described with respect to FIG. 8, may be formed or placed on an inner surface of cover 1142, or within base 1140. In some examples, a battery or other power source of IMD 10B may be included within base 1140. In the illustrated example, antenna 26B is formed or placed on the outer surface of cover 1142, but may be formed or placed on the inner surface in some examples. In some examples, insulative cover 1142 may be positioned over an open base 1140 such that base 1140 and cover 1142 enclose the circuitries and other components and protect them from fluids such as body fluids. The housing including base 1140 and insulative cover 1142 may be hermetically sealed and configured for subcutaneous implantation.

Circuitries and components may be formed on the inner side of insulative cover 1142, such as by using flip-chip technology. Insulative cover 1142 may be flipped onto a base 1140. When flipped and placed onto base 1140, the components of IMD 10B formed on the inner side of insulative cover 1142 may be positioned in a gap 1144 defined by base 140. Electrodes 16C and 16D and antenna 26B may be electrically connected to circuitry formed on the inner side of insulative cover 1142 through one or more vias (not shown) formed through insulative cover 1142. Insulative cover 1142 may be formed of sapphire (i.e., corundum), glass, parylene, and/or any other suitable insulating material. Base 1140 may be formed from titanium or any other suitable material (e.g., a biocompatible material). Electrodes 16C and 16D may be formed from any of stainless steel, titanium, platinum, iridium, or alloys thereof. In addition, electrodes 16C and 16D may be coated with a material such as titanium nitride or fractal titanium nitride, although other suitable materials and coatings for such electrodes may be used.

In the example shown in FIG. 11B, the housing of IMD 10B defines a length L, a width W and thickness or depth D and is in the form of an elongated rectangular prism wherein the length L is much larger than the width W, which in turn is larger than the depth D, similar to IMD 10A of FIG. 11A. For example, the spacing between proximal electrode 26C and distal electrode 26D may range from 5 mm to 50 mm, from 30 mm to 50 mm, from 35 mm to 45 mm, and may be any single spacing or range of spacings from 5 mm to 50 mm, such as approximately 40 mm. In addition, IMD 10B may have a length L that ranges from 5 mm to about 70 mm. In other examples, the length L may range from 30 mm to 70 mm, 40 mm to 60 mm, 45 mm to 55 mm, and may be any single length or range of lengths from 5 mm to 50 mm, such as approximately 45 mm. In addition, the width W may range from 3 mm to 15 mm, 5 mm to 15 mm, 5 mm to 10 mm, and may be any single width or range of widths from 3 mm to 15 mm, such as approximately 8 mm. The thickness or depth D of IMD 10B may range from 2 mm to 15 mm, from 5 mm to 15 mm, or from 3 mm to 5 mm, and may be any single depth or range of depths between 2 mm and 15 mm, such as approximately 4 mm. IMD 10B may have a volume of three cubic centimeters (cm) or less, or 1.5 cubic cm or less, such as approximately 1.4 cubic cm.

In the example shown in FIG. 11B, once inserted subcutaneously within the patient, outer surface of cover 1142 faces outward, toward the skin of the patient. In addition, as shown in FIG. 11B, proximal end 1146 and distal end 1148 are rounded to reduce discomfort and irritation to surrounding tissue once inserted under the skin of the patient. In addition, edges of IMD 10B may be rounded.

The following numbered clauses illustrate one or more aspects of the devices and techniques described in this disclosure.

Clause 1. A device comprising: a memory; and processing circuitry coupled to the memory and configured to: receive medication data for a patient, the medication data being indicative of compliance of the patient with a prescribed medication plan; determine a compliance metric for the patient with the prescribed medication plan based on the medication data, wherein the compliance metric is indicative of a degree of compliance of the patient with the patient with the prescribed medication plan; obtain, via one or more medical devices, values for one or more physiological parameters for the patient; and based on the compliance metric and the values for the one or more physiological parameters for the patient, determine a heart failure risk score for the patient.

Clause 2. The device of clause 1, wherein to determine the compliance metric for the patient with the prescribed medication plan based on the medication data, the one or more processors are configured to determine the compliance metric based on a frequency with which the patient takes the medication relative to an expected frequency established by the prescribed medication plan.

Clause 3. The device of clause 1 or 2, wherein to determine the compliance metric for the patient with the prescribed medication plan based on the medication data, the one or more processors are configured to determine the compliance metric based on a deviation from times in which the patient takes the medication relative to expected times established by the prescribed medication plan.

Clause 4. The device of any of clauses 1-3, wherein to determine the compliance metric for the patient with the prescribed medication plan based on the medication data, the one or more processors are configured to determine the compliance metric based on an amount of medication taken during a time period relative to an expected amount of medication taken during the time period as established by the prescribed medication plan.

Clause 5. The device of any of clauses 1-4, wherein the compliance metric comprises a diagnostic state selected from a group of diagnostic states.

Clause 6. The device of any of clauses 1-5, wherein the one or more physiological parameters include a subcutaneous tissue impedance parameter.

Clause 7. The device of any of clauses 1-6, wherein the processing circuitry is further configured to: determine a plurality of heart failure risk scores for the patient, each heart failure risk score being associated with a first corresponding timestamp; determine a plurality of compliance metrics for the patient, each compliance metric being associated with a second corresponding timestamps; and match, using the first corresponding timestamps and the second corresponding timestamps, the heart failure risk scores to temporally correlated compliance metrics.

Clause 8. The device of clause 7, wherein the processing circuitry is further configured to compare a trend of the plurality of heart failure risk scores for the patient to a trend of the temporally correlated plurality of amounts of compliance for the patient to determine an effect that a level of compliance of the patient with the prescribed medication plan is having on a health status of the patient, wherein the health status of the patient is a function of the plurality of heart failure risk scores.

Clause 9. The device of clause 7, wherein the processing circuitry is further configured to, based on the heart failure risk scores and the temporally correlated amounts of compliance, determine whether a level of compliance of the patient with the prescribed medication plan is affecting a health status of the patient, wherein the health status of the patient is a function of the plurality of heart failure risk scores.

Clause 10. The device of clause 7, wherein the processing circuitry is further configure to, based on the heart failure risk scores and the temporally correlated amounts of compliance, determine that a level of compliance of the patient with the prescribed medication plan is adversely affecting the health status of the patient, wherein the health status of the patient is a function of the plurality of heart failure risk scores; and send a notification to another device indicating that the level of compliance of the patient with the prescribed medication plan is adversely affecting the health status of the patient.

Clause 11. The device of clause 7, wherein the processing circuitry is further configure to: based on the heart failure risk scores and the temporally correlated amounts of compliance, determine that a level of compliance of the patient with the prescribed medication plan is adversely affecting the health status of the patient; and in response to determining that the level of compliance of the patient with the prescribed medication plan is adversely affecting the health status of the patient, modifying the prescribed medication plan.

Clause 12. The device of clause 7, wherein the processing circuitry is further configure to: based on the heart failure risk scores and the temporally correlated amounts of compliance, determine that a level of compliance of the patient with the prescribed medication plan is positively affecting the health status of the patient; and send a notification to another device.

Clause 13. The device of any of clauses 1-12, wherein to determine the heart failure risk score for the patient, the one or more processors are configured to input the compliance metric and the values for the one or more physiological parameters for the patient into a probability model.

Clause 14. The device of clause 13, wherein the probability model comprises a Bayesian network.

Clause 15. The device of any of clauses 1-14, wherein the processing circuitry is further configured to generate an alert in response to the heart failure risk score meeting or exceeding a threshold.

Clause 16. The device of clause 15, wherein the processing circuitry is further configured to generate the alert in response to determining that a change in the heart failure risk score for the patient correlates to a lack of compliance of the patient with the prescribed medication plan.

Clause 17. The device of any of clauses 1-16, wherein to obtain the medication data for the patient, the processing circuitry is configured to solicit user input indicating the compliance with the prescribed medication plan.

Clause 18. The device of any of clauses 1-17, wherein the one or more medical devices include an implantable medical device.

Clause 19. The device of clause 18, wherein the implantable medical device includes an implantable cardiac monitoring devices.

Clause 20. A method comprising: receiving medication data for a patient, the medication data being indicative of compliance of the patient with a prescribed medication plan; determining a compliance metric for the patient with the prescribed medication plan based on the medication data, wherein the compliance metric is indicative of a degree of compliance of the patient with the patient with the prescribed medication plan; obtaining, via one or more medical devices, values for one or more physiological parameters for the patient; and based on the compliance metric and the values for the one or more physiological parameters for the patient, determining a heart failure risk score for the patient.

Clause 21. The method of clause 20, wherein determining the compliance metric for the patient with the prescribed medication plan based on the medication data comprises determining the compliance metric based on a frequency with which the patient takes the medication relative to an expected frequency established by the prescribed medication plan.

Clause 22. The method of clause 20 or 21, wherein determining the compliance metric for the patient with the prescribed medication plan comprises determining the compliance metric based on a deviation from times in which the patient takes the medication relative to expected times established by the prescribed medication plan.

Clause 23. The method of any of clauses 20-22, wherein determining the compliance metric for the patient with the prescribed medication plan based on the medication data comprises determining the compliance metric based on an amount of medication taken during a time period relative to an expected amount of medication taken during the time period as established by the prescribed medication plan.

Clause 24. The method of any of clauses 20-23, wherein the compliance metric comprises a diagnostic state selected from a group of diagnostic states.

Clause 25. The method of any of clauses 20-24, wherein the one or more physiological parameters include a subcutaneous tissue impedance parameter.

Clause 26. The method of any of clauses 20-25, further comprising: determining a plurality of heart failure risk scores for the patient, each heart failure risk score being associated with a first corresponding timestamp; determining a plurality of compliance metrics for the patient, each compliance metric being associated with a second corresponding timestamps; and matching, using the first corresponding timestamps and the second corresponding timestamps, the heart failure risk scores to temporally correlated compliance metrics.

Clause 27. The method of clause 26, further comprising: based on the heart failure risk scores and the temporally correlated amounts of compliance, determining that a level of compliance of the patient with the prescribed medication plan is adversely affecting the health status of the patient, wherein the health status of the patient is a function of the plurality of heart failure risk scores; and sending a notification to another device indicating that the level of compliance of the patient with the prescribed medication plan is adversely affecting the health status of the patient.

Clause 28. The method of any of clauses 20-27, wherein determining the heart failure risk score for the patient comprises inputting the compliance metric and the values for the one or more physiological parameters for the patient into a probability model.

Clause 29. The method of clause 28, wherein the probability model comprises a Bayesian network.

Clause 30. A computer-readable storage medium storing instructions that when executed by one or more processors cause the one or more processors to perform the method of any of clauses 20-29.

The techniques described in this disclosure may be implemented, at least in part, in hardware, software, firmware, or any combination thereof. For example, various aspects of the techniques may be implemented within one or more microprocessors, DSPs, ASICS, FPGAs, or any other equivalent integrated or discrete logic QRS circuitry (as in QRS complex), as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, ROM, NVRAM, DRAM, SRAM, Flash memory, magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD or other medical device, an external programmer, a combination of a medical device and external programmer, an integrated circuit (IC) or a set of ICs, and/or discrete electrical circuitry, residing in a medical device and/or external programmer.

Furthermore, although described primarily with reference to examples that provide a probability score to indicate worsening heart failure, other examples may additionally or alternatively automatically modify a therapy in response to the probability score exceeding a predetermined threshold. The therapy may be, as examples, a substance delivered by an implantable pump, cardiac resynchronization therapy, refractory period stimulation, or cardiac potentiation therapy. These and other examples are within the scope of the following claims.

1. A device comprising:
a memory; and
processing circuitry coupled to the memory and configured to:
receive medication data for a patient, the medication data being indicative of compliance of the patient with a prescribed medication plan;
determine a plurality of values for a compliance metric for the patient with the prescribed medication plan based on the medication data, wherein the compliance metric is indicative of a degree of compliance of the patient with the patient with the prescribed medication plan;
obtain, via one or more medical devices, values for one or more physiological parameters for the patient;
based on a plurality of values for the compliance metric and the values for the one or more physiological parameters for the patient, determine a plurality of heart failure risk scores for the patient; and
based on the plurality of heart failure risk scores and temporally correlated values of the plurality of values for the compliance metric, determine that a level of compliance of the patient with the prescribed medication plan is adversely affecting a health status of the patient, wherein the health status of the patient is a function of the plurality of heart failure risk scores.

2. The device of claim 1, wherein the processing circuitry is configured to determine the compliance metric based on a frequency with which the patient takes the medication relative to an expected frequency established by the prescribed medication plan.

3. The device of claim 1, wherein the processing circuitry is configured to determine the compliance metric based on a deviation from times in which the patient takes the medication relative to expected times established by the prescribed medication plan.

4. The device of claim 1, wherein the processing circuitry is configured to determine the compliance metric based on an amount of medication taken during a time period relative to an expected amount of medication taken during the time period as established by the prescribed medication plan.

5. The device of claim 1, wherein the compliance metric comprises a diagnostic state selected from a group of diagnostic states.

6. The device of claim 1, wherein the one or more physiological parameters include a subcutaneous tissue impedance parameter.

7. The device of claim 1, wherein the processing circuitry is further configured to:
determine the plurality of heart failure risk scores for the patient with each heart failure risk score being associated with a first corresponding timestamp;
determine the plurality of values for the compliance metric for the patient with each compliance metric being associated with a second corresponding timestamps; and
match, using the first corresponding timestamps and the second corresponding timestamps, the heart failure risk scores to the temporally correlated values of the plurality of values for the compliance metric.

8. The device of claim 7, wherein the processing circuitry is further configured to:
compare a trend of the plurality of heart failure risk scores for the patient to a trend of the temporally correlated values of the plurality of values for the compliance metric to determine an effect that the level of compliance of the patient with the prescribed medication plan is having on the health status of the patient.

9. (canceled)

10. (canceled)

11. The device of claim 7, wherein the processing circuitry is further configured to:
in response to determining that the level of compliance of the patient with the prescribed medication plan is adversely affecting the health status of the patient, modifying the prescribed medication plan.

12. The device of claim 7, wherein the processing circuitry is further configured to:
based on the heart failure risk scores and the temporally correlated values of the plurality of values for the compliance metric, determine that a level of compliance of the patient with the prescribed medication plan is positively affecting the health status of the patient; and
send a notification to another device.

13. The device of claim 1, wherein to determine the heart failure risk score for the patient, the processing circuitry is further configured to input the compliance metric and the values for the one or more physiological parameters for the patient into a probability model.

14. The device of claim 13, wherein the probability model comprises a Bayesian network.

15. The device of claim 1, wherein the processing circuitry is further configured to generate an alert in response to the heart failure risk score meeting or exceeding a threshold.

16. The device of claim 15, wherein the processing circuitry is further configured to generate the alert in response to determining that a change in the heart failure risk score for the patient correlates to a lack of compliance of the patient with the prescribed medication plan.

17. The device of claim 1, wherein to obtain the medication data for the patient, the processing circuitry is configured to solicit user input indicating the compliance with the prescribed medication plan.

18. The device of claim 1, wherein the one or more medical devices include an implantable medical device.

19. The device of claim 18, wherein the implantable medical device includes an implantable cardiac monitoring device.
